(19) United States
(12) Patent Application Publication
(10) Pub. No.: US 2006/0067924 A1
(43) Pub. Date: Mar. 30, 2006

(54) Probiotic bacteria and methods

(76) Inventors: Margie D. Lee, Watkinsville, GA (US); Barry G. Harmon, Athens, GA (US); Charles L. Hofacre, Watkinsville, GA (US)

Correspondence Address: GREENLEE WINNER AND SULLIVAN P C, 4875 PEARL EAST CIRCLE, SUITE 200, BOULDER, CO, 80301, US

(21) Appl. No.: 11/273,617

(22) Filed: Nov. 14, 2005

Related U.S. Application Data
(63) Continuation-in-part of application No. PCT/US04/15378, filed on May 14, 2004.
(60) Provisional application No. 60/470,807, filed on May 14, 2003.

(51) Int. Cl. A61K 35/74; C12Q 1/04

(52) U.S. Cl. 424/93.45; 435/34

(57) ABSTRACT

Provided herein are molecular methods for assessing the state of gastrointestinal microflora of an animal, especially a species of poultry, and methods for identifying probiotic bacteria by comparing certain bacteria present in animals fed a diet not containing antibiotics but absent or present in significantly lower numbers in animals fed a diet containing antibiotics.

### CROSS REFERENCE TO RELATED APPLICATIONS

This application is a Continuation-in-Part of International Application PCT/US04/15378, filed May 14, 2004, which claims benefit of U.S. Provisional Application 60/470,807, filed May 14, 2003.

### ACKNOWLEDGMENT OF FEDERAL RESEARCH SUPPORT

This invention was made, at least in part, with funding from the United States Department of Agriculture (Grant No. USDA-1433 Formula Funds). Accordingly, the United States Government has certain rights in this invention.

### BACKGROUND OF INVENTION

This invention is in the field of agriculture, in particular, as related to methods for identifying probiotic bacteria for use in dietary supplements for poultry, to methods for improving poultry health, performance and product safety through the use of probiotic dietary supplements and to methods for assessing the desirability of the microbial population of the gastrointestinal tract of poultry, especially in birds fed with antibiotic-supplemented feed.

Nearly 100% of chickens receive diets containing antibiotic drugs during some part of production. (National Research Council, Washington, D.C., National Academy Press, 1999). There is growing concern regarding the use of antibiotics in chicken and other poultry feed due to development of antibiotic resistance by bacteria in that environment. Therefore, Europe has banned the use of antibiotics in chicken feed, and there is movement to ban their use in the United States. However, antibiotic supplemented feed is associated with growth promotion and disease prevention, so removal of antibiotics without a suitable substitute will have a negative impact on the animal production industry. There are currently no alternative means to replace the economic advantages of growth-promoting antibiotics. The cost of such a ban to the chicken broiler industry has been estimated to be between $283 and $572 million dollars per year. (NRC, 1999; Food and Agricultural Policy Research Institute, U.S. Agricultural Outlook, Staff Report #1-98. Ames, Iowa: Iowa State University).

It has long been known that densely colonized intestinal bacteria play an important role in the health and performance through their effect on gut morphology, nutrition, and pathogenesis of intestinal disease and immune response. Intestinal bacteria are primarily responsible for degrading the copious amounts of mucus produced by goblet cells in the intestinal mucosa (Falk et al. 2000. Microbiol Mol. Biol. Rev. 62:1157-70). Certain of the microbial flora are also believed to protect against colonization of the gastrointestinal tract by pathogens and to stimulate the immune response in the gut (Mead, 1989, J. Exp. Zool. Suppl. 3:48-54).

Studies based on the culturable bacteria flora of chickens have been extensively conducted (Rolfe 1991. J Nutr. 130(Supp): 396S402S). The predominant bacteria present in the chicken ceca are obligate anaerobes (10¹¹ per g) (Barnes, 1972, Am. J. Clin. Nutr. 25:475-79; Barnes, et al. (1972) Am. J. Clin. Nutr. 25:1475-1497; Barnes et al. (1972) Br. Poult. Sci. 13:311-326; Barnes and Impey (1972) J. Appl. Bacteriol. 35:241-251). There have been at least 38 different types of anaerobic bacteria isolated from the chicken ceca (Barnes et al., 1972 supra) with more than 200 total bacterial strains isolated (Mead, 1989. supra). Mead found the gram positive cocci (Peptostreptococcus, etc.) were 28% of the total viable bacteria, Bacteroidaceae (20%), Eubacterium spp. (16%), Bifidobacterium spp. (9%), budding cocci (6%), Gemmiger formicilis (5%), Clostridium spp. (5%) and miscellaneous (11%) (Mead, 1989. supra). However, not all bacteria are culturable; it is estimated that from less than 10% (Amann et al., 1995, Microbiol. Rev. 59:143-169) to about 60% of the bacteria in the chicken cecum grew in culture (Barnes et al. 1972, Br. Poult. Sci. 13: 311-326; Barnes, 1972, Am. J. Clin. Nutr. 25: 1475-1479; Salanitro 1974, Appl. Microbiol. 27: 678-687; Salanitro, J. P. et al. 1974. Appl. Microbiol. 28:439-47). Netherwood et al., Appl. Environ. Microbiol. 65:5134-5138 (1999) used hybridization methods to monitor the response of bacterial flora in the chicken cecum to probiotics, and diet related differences were analyzed by Apajalahti et al., Appl. Environ. Microbiol. 64:4084-4088 (1998) based on a percent G+C profiling. These studies demonstrated that many of the 16S rDNA sequences found in the chicken cecum were not closely related to any previous known bacterial genera. Zhu et al., Appl. Environ. Microbiol. 68:124-137 (2002) isolated 243 unique partial 16S rRNA gene sequences from DNA isolated from the cecal content and the cecal mucosa.

There is need in the art for safe substitutes for antibiotics from poultry feed, especially chicken feed, to prevent antimicrobial resistance and antibiotic-resistant food borne pathogens, while maintaining the beneficial effects of antibiotic administration, including increased weight gain, feed conversion and disease prevention, and thus better economics of meat, dairy and egg production in animals, including birds such as poultry, and especially in chickens. The present invention meets this need by replacing antibiotics with prebiotics and/or probiotics, so that the intestinal microbiota is similar to that of birds not fed antibiotic supplements. There is also a need in the art for methods by which prebiotics and probiotics can be identified by measuring the microflora in the gastrointestinal tract or feces of an animal, especially poultry, and in particular, chickens.

### SUMMARY OF THE INVENTION

This invention provides a method for evaluating the changes in the intestinal microbial flora of animals, e.g., poultry, especially chickens, resulting from growth-promoting antibiotic feed or probiotic-supplemented feed. By comparing the intestinal microbial flora of antibiotic-supplemented and control (no antibiotic) animals, prebiotics and probiotic microorganisms, especially bacteria, are identified. The animal can be mammal, reptile, amphibian or bird. The molecular methods by which gut microflora are analyzed yield a more complete picture of gastrointestinal tract microflora, including relative proportions of different bacteria. This method allows the identification of bacteria or other microorganisms appropriate for use as a probiotic dietary supplement for animals including, but not limited to, birds, e.g., poultry, especially chickens. In this manner, advantageous growth rate and feed efficiency, and thus profit, are matched without the need for antibiotics to manipulate the intestinal flora of the animal of interest. The microflora can be analyzed using fecal samples from the animal of interest or using samples obtained from particular portions of the gastrointestinal tract.

In addition, the methods of the present invention can be employed to predict or diagnose intestinal disease or assess the health of the gastrointestinal tract prior to the clinical manifestation of symptoms. The use of the probiotic bacteria described herein in dietary supplements for animals such as birds and poultry, especially chickens, results in reduced colonization of the gastrointestinal tracts of poultry by pathogens, including but not limited to Clostridium perfringens, Salmonella spp. and Campylobacter spp. Probiotic bacteria of the present invention include Clostridium irregularis (also called C. irregulars), Clostridium lituseburense and Clostridium disporicum. Clostridium irregularis is available from the American Type Culture Collection (ATCC), Manassas, Va., Accession No. 25756. Clostridium lituseburense is available from the ATCC under Accession No. 25759, and Clostridium disporicum is available from the ATCC under Accession No. 43838. One or more of the following bacteria can also be used as probiotics: Lactobacillus crispatus, Lactobacillus delbreukii, Lactobacillus salivarius, Lactobacillus aviarius, and Lactobacillus reuteri. Lactobacillus acidophilus is well known for its beneficial qualities.

This invention further provides molecular techniques to identify the microbial, especially bacterial, species or genera and to determine community succession in the gastrointestinal tract or a portion thereof in an animal, i.e., a mammal, a reptile, an amphibian or a bird, as specifically exemplified, in the ileum of poultry, e.g., chickens, fed a particular diet, for example, a corn-soy diet lacking coccidiostats and growth-promoting antibiotics. These findings enable ways to achieve economically advantageous growth rate and feed efficiency and/or improved general health, without use of antibiotics by manipulation of the intestinal flora by feeding viable cells of probiotic bacteria including, but not limited to, C. perfringens, Salmonella spp. and/or Campylobacter spp.

The present invention also provides methods to predict intestinal disease prior to the clinical manifestation of symptoms and methods to prevent colonization of pathogens, such as C. perfringens, Salmonella spp. or Campylobacter spp, for example.

The methods of the present invention using 16S rRNA gene-based data provide a more accurate and representative measure of the true population of intestinal microflora than culture-based ones due to the difficulties in growing the microorganisms, many of which are fastidious in their nutritional requirements or obligately anaerobic, from the gastrointestinal tracts of mammals or birds, such as poultry, and in particular, chickens. Fecal samples or samples taken directly from the gastrointestinal tract can serve as the source of microorganisms for analysis.

It is a further object of the invention to provide a probiotic composition for use in mammals, reptiles, amphibians, birds, poultry and especially chickens, containing at least one nonpathogenic, gastrointestinal tract-colonizing species selected from the group consisting of Clostridium irregularis (also called C. irregulars), Clostridium lituseburense and Clostridium disporicum. The probiotic composition of the present invention does not require the presence of a Lactobacillus, for example, L. acidophilus, which is commonly present in probiotic compositions, although at least one Lactobacillus noted above can be used.

Also within the scope of the present invention are methods for improving the general health, promoting growth and/or reducing the incidence of pathogenic microorganisms which colonize the gastrointestinal tract of a mammal, bird, poultry or chicken in which the animal of interest receives (per os in feed, dietary supplement or drinking water) a probiotic composition comprising viable cells of at least one species selected from the group consisting of Clostridium irregularis (also called C. irregulars), Clostridium lituseburense and Clostridium disporicum in an amount effective to colonize at least one region of the gastrointestinal tract of the mammal, bird, poultry or chicken. The probiotic composition does not include L. acidophilus, although one or more other Lactobacillus species (reuteri, delbreukii, crispatus, salivarius or aviarius) can be incorporated.

Although previous studies have documented the variation or effects of some aspects of intestinal bacteria based on cultivation, a well-designed experiment on different diets using recently developed molecular methods is necessary to correctly and accurately monitor the intestinal bacterial flora.

### BRIEF DESCRIPTION OF THE DRAWINGS

FIG. 1 shows the phylogeny of bacteria commonly found in chicken intestine.

FIG. 2 is a comparison of T-RFs of amplified 16S gene between control and treatments at different ages.

FIG. 3 is the distribution of the bacterial main genera or groups present in the Gr1 (fed with ad libitum commercial corn-soy as a control), Gr2 (wheat-based diet), Gr3 (fed with corn-soy plus Aviguard (freeze-dried competitive exclusion product, Bayer plc, Suffolk, England) Aviguard is a, dried competitive exclusion product of Bayer Animal Health), Gr4 (fed with corn-soy plus growth promotant diet), and Gr5 (corn-soy plus monensin).

FIG. 4 is the coverage estimation and number of unique sequences obtained by direct community analysis of pooled sequences from chicken ileum.

FIG. 5 is the identity (percentage) of the total number of sequences present in the chicken ileum.

FIG. 6 is the distribution of bacterial phylogenetic groups or subdivisions in chicken ileum as a function of chicken age.

FIG. 7 is a phylogenetic tree showing 16S rDNA sequences from chicken ileum samples for low G+C-content bacteria. The tree was constructed by neighbor-joining analysis of a distance matrix obtained from a multiple-sequence alignment. Bootstrap values (expressed as percentages of 100 replications) are shown at branch points: values under 50 were not considered significant. The names and GenBank accession numbers for the most related sequences are listed and presented in the Sequence Listing. LBARR16SAZ is SEQ ID NO:1, AB007908 is SEQ ID NO:2, AF257097 is SEQ ID NO:3, LHA306298 is SEQ ID NO:4, AJ420801 is SEQ ID NO:5, AF061009 is SEQ ID NO:6, AB002519 is SEQ ID NO:7, AF089108 is SEQ ID NO:8, AB001936 is SEQ ID NO:9, Y2669.1 is SEQ ID NO:10, and AY007244 is SEQ ID NO:11.

FIG. 8 shows distribution of bacterial composition as detected by T-RFLP analysis with different diets.

FIG. 9 shows the distribution of bacteria as varied according to diet and chicken age.

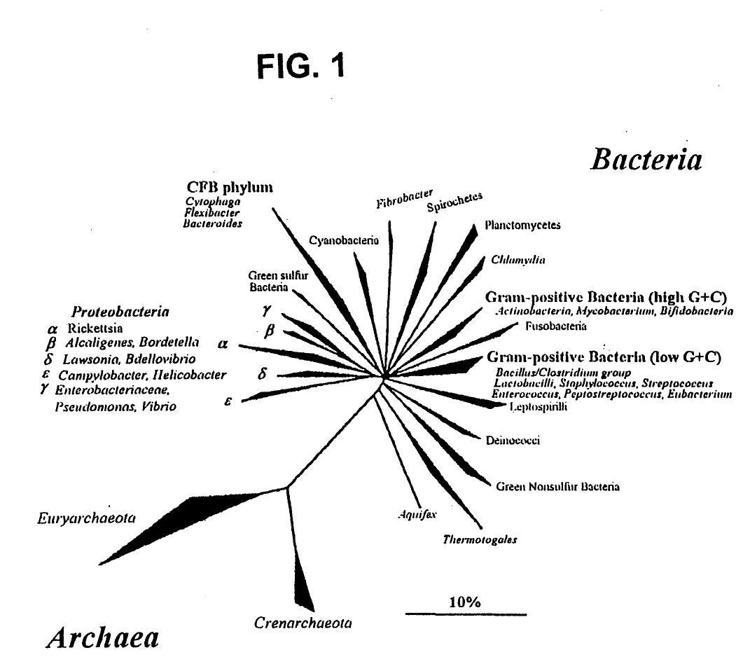

### DETAILED DESCRIPTION OF THE INVENTION

Probiotic is used herein to describe bacteria isolated from a natural source and having the property of inhibiting growth of pathogenic microorganisms in an animal, a mammal, reptile, amphibian, a bird, poultry and especially chickens, for example, C. perfringens, in the context of the gastrointestinal tract of poultry, e.g., chickens. Probiotic bacteria are selected by comparing the microflora of the animal of interest administered one or more antibiotics to the intestinal microflora of the animal not administered any antibiotics.

Prebiotic is used herein to describe compounds, usually oligosaccharides, which promote the growth of beneficial bacteria, especially in the gastrointestinal tract of an animal, a mammal, reptile, amphibian or bird such as poultry, especially chickens.

As used herein, nonpathogenic means that the microorganism, for example, a bacterium, is neither pathogenic to humans nor the animal of interest. The microorganism does not cause disease in the human or animal.

Poultry includes, without limitation, chickens, ducks, geese, turkeys and guinea fowl.

In the present context, gastrointestinal tract-colonizing means that a microorganism, especially a bacterium, binds to and multiplies on the surface of tissue in the lumen of the gastrointestinal tract or a portion thereof of the animal of interest. Portions of interest as exemplified herein include the cecum and the ileum of a chicken.

As used herein, antibiotic fed animals are those fed a diet (or water) into which at least one antibiotic is incorporated. No-antibiotic-fed animals are those supplied with diet and with water, neither of which comprises an antibiotic.

The descriptions provided herein are for illustrative purposes, and are not intended to limit the scope of the invention as claimed. Any variations in the exemplified methods that occur to the skilled artisan are intended to fall within the scope of the present invention.

The model animal discussed herein is the chicken. The microbial ecology of the chicken small intestine is relatively poorly defined, primarily because studies have focused on the cecum. In order to better understand the ecology of this environment, we used 16S ribosomal DNA gene sequencing to identify the dominant members of the bacterial flora from different age chickens. More than 68.85% of sequences, at all the tested ages, were related to those of Lactobacillus. Several sequences were identified in the library for bacteria associated with disease in humans and poultry such as clostridia, Campylobacter and staphylococci. However, the sequences of bacterial populations varied significantly by age of the birds. At all ages, sequences were identified in the library showing homology to the genus Clostridium. There was a unique community structure at 3 days of age with the sequences homologous to culturable bacteria such as L. delbrueckii, C. perfringens and Campylobacter coli. From 7 days of age to 21 days, a similar community structure was maintained with dominant sequences related to L. acidophilus, Enterococcus and Streptococcus. To some extent the bacterial community at 49 days of age was similar to those at age 28, with the abundant sequences homologous to L. crispatus, but it was significantly different from those of other ages.

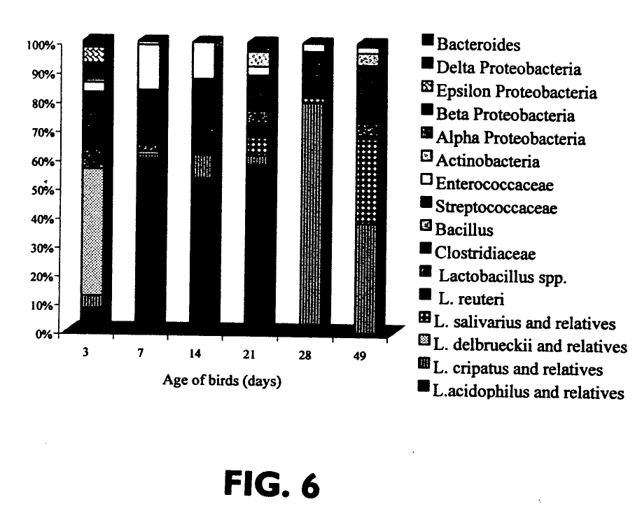

A molecular ecological approach was used to identify the bacterial composition and to determine community succession in the ileum of chickens fed a corn-soy diet lacking coccidiostats and growth-promoting antibiotics. We isolated random clones of 16S ribosomal DNA gene sequences after multiple PCR amplification of bacterial genomic DNA isolated from the ileum of chickens at 3, 7, 14, 21, 28 and 49 days of age. From analysis of 614 clones isolated from the 16S rDNA libraries, we identified four major phyla. These phyla included low and high G+C gram-positives, proteobacteria and the CFB group (Table 4 and FIG. 6). Eleven families or groups and sixteen genera were identified among the 16S rDNA sequences analyzed. The bacterial microbiota consisted predominantly of low G+C gram-positive bacteria, whose representative distinct sequences were shown in FIG. 6, with Lactobacillus accounting for 68.85% of the total 16S rDNA sequences in the libraries. The low G+C gram-positives consisted of five families or groups represented by nine genera. Identification of members of dominant genera Lactobacillus, Enterococcus and Streptococcus were culturable and have been often isolated from normal ileum (Salanitro, J. P. et al. 1978. Appl. Environ. Microbiol. 35:782-90). However, we did not expect to find that Clostridium was a dominant group at age 3 and age 49 in the ileum according to previous studies (Barnes et al. 1972; Salanitro, 1978. supra). We detected Clostridium spp. in the ileal flora at all ages. Stutz and Lawton (1984) reported detection of clostridia, including C. perfringens, by culture of the ileum of 2-day-old chicks (Stutz, M. W. and G. C. Lawton, 1984, Poult. Sci. 63:2241-6). About 15% of our total sequences at 3 days of age had homology to C. perfringens, which is an important cause of necrotic enteritis in broilers (George, B. A. et al. 1982, Poult. Sci. 61:447450; Long, J. R. 1973, Can. J. Comp. Med. 37:302-308). We also detected sequences of segmented, filamentous Clostridium spp., commonly found in healthy animals, at 14 days of age (Snel, J. et al. 1995, Int. J. Syst. Bacteriol. 45:780-2).

There are various formulations of antibiotics used as growth promotants. In the United States many companies use virginiamycin in the grower and finisher feed for broiler chickens. In order to determine its effect on the ileum microflora, we sequenced 16S rDNA genes isolated from libraries prepared from these birds at 28 and 49 days of age. Birds fed virginiamycin contained significantly fewer Lactobacillus species in the ileum than controls at both ages. In addition, the ratios among the dominant Lactobacillus species and the dominant Clostridium species were different. Changes in the other bacterial populations appeared to be minor.

This invention allows us to achieve present day growth rate and feed efficiency without using antibiotics by manipulation of the intestinal flora. The invention is used to predict intestinal disease prior to the clinical manifestation of symptoms and to employ methods that prevent colonization of pathogens, such as, C. perfringens, Salmonella spp. or Campylobacter spp.

A comparative study of bacterial community of the chicken ileum was carried out using 16S rDNA gene analysis. The intestinal microbiota is part of a complex ecosystem. This study examined the effect of the growth promoting antibiotic, virginiamycin, and other commercial diets on the distribution and community structure of intestinal bacterial flora. Bacterial communities in the intestines of chickens were compared using terminal restriction fragment length polymorphism (T-RFLP) analysis targeting the 16S ribosomal DNA combining with 16S rDNA cloning library. The chickens were fed 4 different diets including a commercial corn-soy diet, corn-soy plus growth promotant diet, corn-soy plus monensin, and a wheat diet. A group was also administrated a probiotic at 1 day age and fed a corn-soy diet. After feeding of the birds with the experimental diets, the differences in the bacterial community structures in the ileum were detected in the form of different profiles of terminal restriction fragments (T-RFs). Some of the T-RFs were commonly distributed, i.e., they were found in all samples, while others varied in distribution and correlated with specific diets. Significant differences were found between the control group (corn-soy diet) and the experimental groups by pairwise-analyzing the T-RFs=profiles. These data indicate that feeding different antimicrobials causes significant alterations in the microbial community structure.

It has been shown that there is a relationship between the intestinal microflora and health of animals (Long et al. 1973. supra). Many strategies are currently being used to strengthen host defenses and improve weight gain by supplementing animal feed with ingredients that promote the growth of beneficial bacteria in the intestine. The common modulators of gastrointestinal tract ecology are probiotics (Netherwood et al. 1999, supra, Rolfe 2000 J. Nutr. 130 (Suppl):396S-402S, Tannock, 2000 Appl. Environ. Microb. 66:2578-2588; Henderics et al. 1982 J. Vet. Med. Suppl. 33:56-63) and growth-promoting antibiotics (George et al; 1982, supra; NRC 1999, Elasser et al. 1997 Comp. Biochem. Physiol A. Physiol. 116:209-211). In order to understand the mechanism of action of these products and to develop more effective products, there is a need to monitor the intestinal microbial community structure. The intestinal microbial flora related to different diets were studied in chickens from the earlier studies based on cultivation-based techniques to the recent molecular technique-based approaches. Diets containing rye or pectin were found to significantly influence the intestinal bacteria composition and metabolic activity of the intestinal microflora (Guslis et al. 1999 J. Food Protec. 62:252-256). Some studies suggested that the intestinal bacterial flora could be managed by the feed gradients conducive to the growth of beneficial intestinal bacteria, as well as direct introduction of bacterial populations that favor good health and nutrition in animals (Garriga et al. 1998 J. Appl. Microbiol. 84:125-132, Jin et al. 1998 Anim. Feed Sci. Technol. 70:197-209). The fact that current agricultural practices in the production of food animals often use antibiotics for the treatment of clinical disease and for prevention of subclinical bacterial and/or coccidial infections led many researchers to study the effects of antibiotics on intestinal microbial flora. The results showed that many of these antibiotics that prevent subclinical infections resulted in enhancement of growth rate and efficiency in utilizing feed and are often referred to as antibiotic growth promotants (AGPs) (George et al., 1982 supra; NRC 1999 supra; Elasser et al., 1997 supra). These AGPs have significant economic benefits for the food animal production industry (Hendericks, 1982, supra). In some instances it has been shown that these AGPs inhibit the growth of specific bacteria such as Clostridium perfringens (George, 1982. supra). However, the actual mode of action for the AGPs has not been determined (Walton 1982 supra; Falk et al. 2000 supra). Since these AGPs are antimicrobial agents, it has been assumed that they might be effective by altering the populations of bacteria in the intestinal flora (Walton 1982 J. Vet. Med. Suppl. 33:77-82; Decuypere et al. 1973 Zb. Bakt. 223:248; Vervaecke et al. 1979 J. Animal Sci. 49:1447).

Although previous studies documented the variation or effects of some aspects of intestinal bacteria based on cultivation, a well-designed experiment on different diets using recently developed molecular methods is necessary to monitor the intestinal bacterial flora. Communities of Bacteria and Archaea have been successfully explored using terminal restriction fragment length polymorphism (T-RFLP) analysis of amplified total community 16S rDNA (Avaniss-Aghajani et al. 1994 BioTechniques 17:144-149; Liu et al. 1997 Appl. Environ. Microbiol. 63:4526-4522; Leser et al. 2000 Appl. Environ. Microbiol. 66:3290-3296), which can provide a rapid and reproducible means to observe bacterial population dynamics and compare community structure under controlled experiments. In this study, we use the T-RFLP analysis combined with 16S rDNA cloning library methods to investigate changes and difference in bacterial community structure in ilea of chickens under a controlled experiment, in which 4 different diets were fed. The aims of this study were to evaluate the impact of different diets, especially those containing antibiotic growth promoters, on the bacterial flora of the chicken ileum.

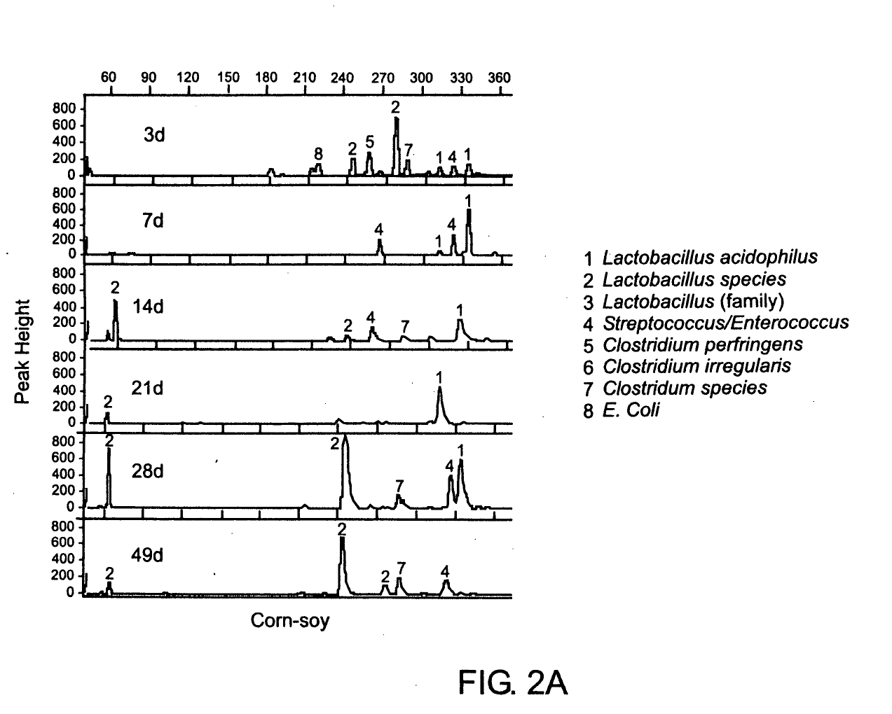

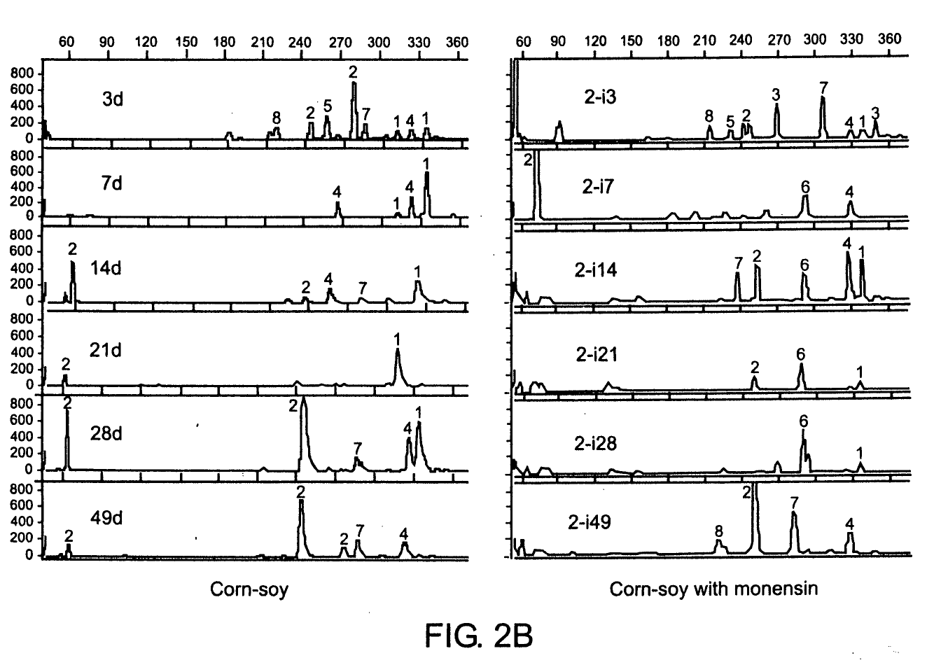

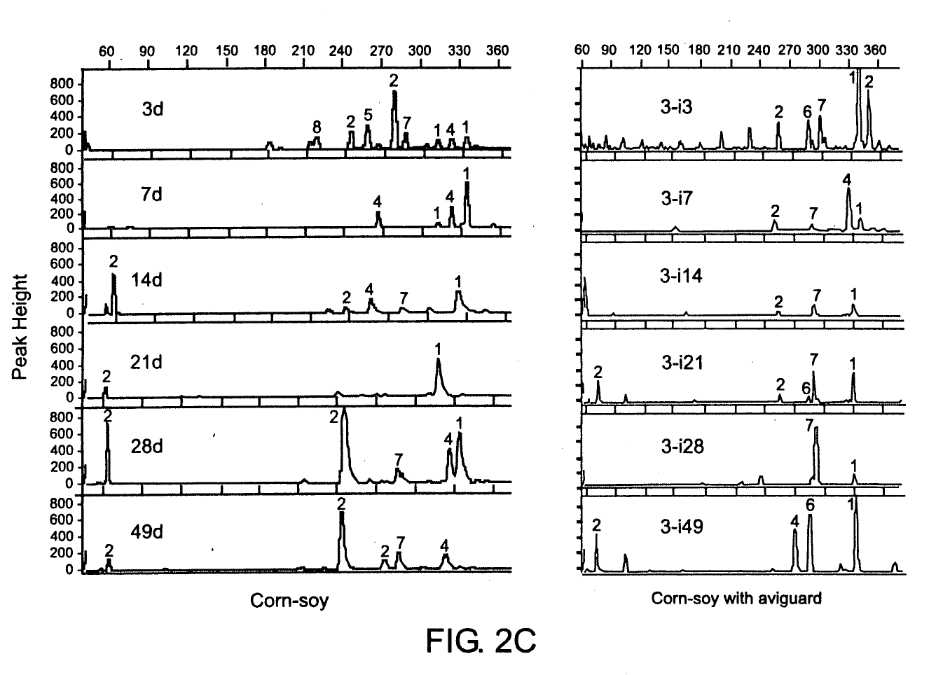

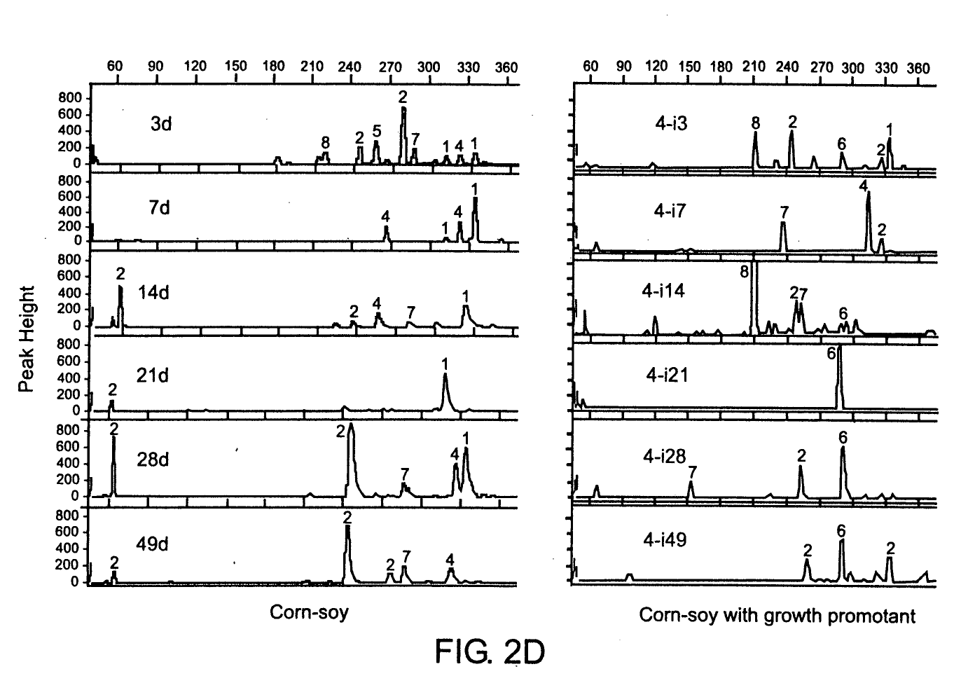

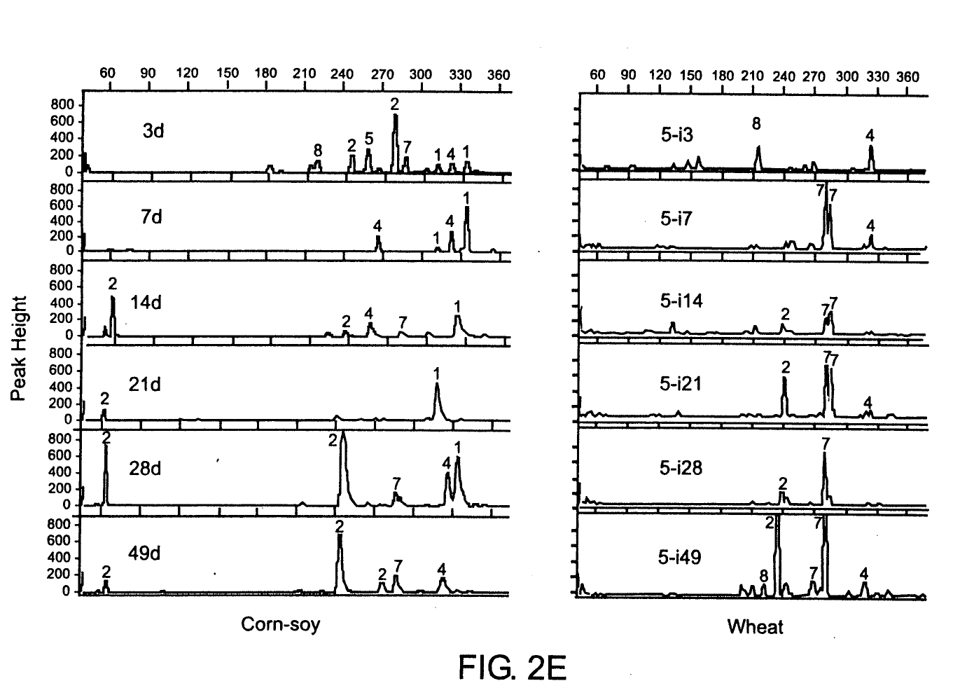

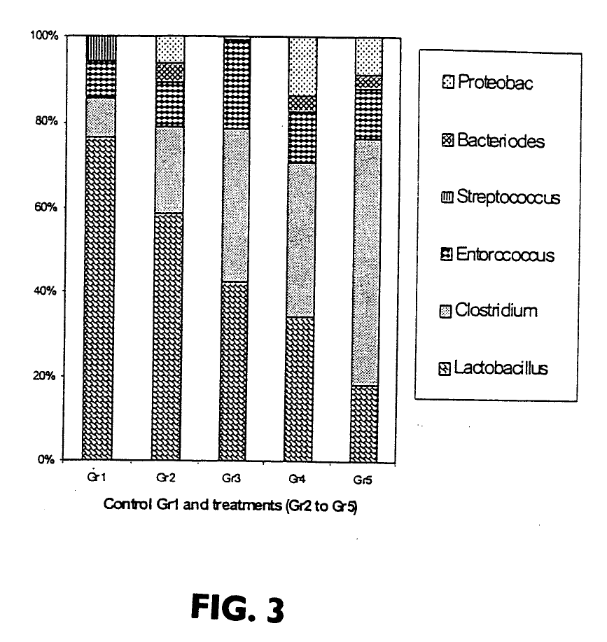

The dominant bacterial microflora were identified in broiler chickens fed different diets: corn-soy feed; corn-soy with monensin (coccidiostat); corn-soy with Aviguard, competitive exclusion product of Bayer Animal Health; corn-soy with growth promoting antibiotics (Starter with BMD and Grower with virginiamycin); wheat feed (see FIG. 2, FIG. 3 and Table 2).

The bacterial populations are identified using genetic analysis of the 16S RNA gene by GeneScan-Terminal Restriction Fragment Length Polymorphism (T-RFLP) using 16S universal primers and cloning and DNA sequencing of 16S PCR products. GeneScan T-RFLP requires the following steps: labeling the PCR product by using labeled primers; digesting the PCR product with restriction enzymes; separating fragments on gel; and detecting terminal fragments. The sizes of terminal fragments can be calculated based on DNA sequence analysis.

The detection for main species was consistent between the cloning library method and TRFLP methods. Combining with the experiment of template ratio vs PCR product ratio, it was shown that the high frequencies or T-RF peak areas of certain species or group were related to its high amount DNA concentrations in the natural samples. TRF pattern analysis allows rapid monitoring of the variations and differences in complex bacterial communities in the gastrointestinal tracts of animals or birds, poultry or the chicken ileum with age and between control and treatment groups.

TABLE 1
PCR product ratios amplified from the template ratios of
# (Lactobacillus acidophilus ATCC 33199) to 2# (Enterococcus
faecium ATCC 19434, 3# (Bacteroides fragilis ATCC 23745
and 4# (Clostridium perfringens ATCC 13124) respectively

| Template | 1# vs 2# | | 1# vs 3# | | 1# vs 4# | |
|---|---|---|---|---|---|---|
| Ratio | Mean | SD | Mean | SD | Mean | SD |
| 1:1 | 0.905 | 0.411 | 0.913 | 0.357 | 0.708 | 0.317 |
| 4:1 | 4.270 | 2.563 | 4.460 | 2.568 | 2.530 | 0.527 |
| 16:1 | 15.167 | 11.215 | 14.833 | 7.360 | 10.417 | 9.330 |

The components mainly consisted of L. acidophilus, L. crispatus, Clostridium irregularis, C. lituseburense, Enterococcus hirae, Enterococcus sp. and Streptococcus sp. in the control and treatment groups. The relative peak areas of Lactobacillus in control group occurred biggest (73.22%), and least in group 5 fed with monensin (19.25%) (FIG. 3). By contrast, the Clostridium peak area, including mainly C. irregularis and C. lituseburense, was smallest in the control group and largest in the group 5. Other bacterial groups did not vary so much among treatments.

It was found that there were quite different bacterial compositions between the 3-day chickens and the samples from other days. Enterococcus as a dominant group occurred in day 7 and day 14 of group 2, group 3 and group 4, but not in the group 5. L. acidophilus as a dominant species found in the control group and 3 treatments but not in the group 5 fed with the wheat.

TABLE 2
Comparisons of main bacterial composition present in TRFLP peaks in ileum of chickens fed different
diets. The orders of bacterial names are according to the relative abundance of peaks, i.e. 100 (peak
areas/total peak areas) in a sample.

| | | | Fed with corn- | Fed with corn- | Fed with corn- |
|---|---|---|---|---|---|
| | Control fed with | | soy and | soy and growth | soy plus |
| Age | only corn | Fed with wheat | aviguard diet | promotants diet | monensin |
| 3 day | Lactobacillus | Eubacteria sp. | L. acidophilus | L. crispatus | E. coli |
| | delbrueckii | Weissella sp. | Weissella sp. | L. acidophilus | Enterococcus sp. |
| | Clostridium | | C. irregularis | E. coli | |
| | perfringens | | | | |
| | L. crispatus | | | | |
| 7 day | L. acidophilus | L. reuteri | Enterococcus sp. | Enterococcus sp. | C. irregularis |
| | Enterococcus sp. | C. irregularis | L. acidophilus | Corynebacterium, | C. lituseburense |
| | Streptococcus sp. | Enterococcus sp. | L. crispatus | lactofermentum | L. acidophilus |
| | | | | L. crispatus | |
| 14 day | L. reuteri | Enterococcus sp. | L. acidophilus | E. coli | C. lituseburense |
| | L. acidophilus | L. crispatus | E. faecium | Enterococcus sp. | C. irregularis |
| | Streptococcus sp. | L. acidophilus | L. crispatus | | Clostridium sp. |
| | | | | | L. crispatus |
| 21 day | L. acidophilus | C. irregularis | C. lituseburense | C. irregularis | C. irregularis |
| | L. reuteri | L. crispatus | L. acidophilus | Bacteroides sp. | C. lituseburense |
| | | L. acidophilus | | | L. crispatus |
| 28 day | L. crispatus | C. irregularis | C. lituseburense | C. irregularis | C. irregularis |
| | L. acidophilus | C. lituseburense | L. crispatus | Bacteroides sp. | L. crispatus |
| | Enterococcus sp. | Bacteroides sp. | | | L. reuteri |
| | | L. acidophilus | | | |
| 49 day | L. crispatus | L. crispatus | L. crispatus | L. aviaries | C. lituseburense |
| | Clostridium sp. | Clostridium sp. | Clostridium sp. | C. irregularis | L. crispatus |
| | Enterococcus sp. | Escherichia coli | Streptococcus sp. | | |
| | | Enterococcus sp. | | | |

The community structure represented by peak numbers and peak areas of each sample were characterized in the diversity index of Shannon-Weaver. The indices ranged from 0.357 to 2.097 with mean 1.191. The highest indices were found in the control group, and then in the group 5, but the indices were least in the group 4 fed with antibiotics (growth promotants). Statistical analysis results suggest that the diet treatments such as the monensin and growth promotants might have affected the microbial community structure.

The population ecology of the microbial flora of the chicken small intestine is ill defined primarily because studies have focused on the cecum. In order to better understand the ecology of this environment, we isolated random clones of 16S ribosomal DNA gene sequences after multiple PCR amplification of bacteria genomic DNA from six different ages of chickens. More than 68.85% of sequences were related to those of Lactobacillus in all the 6 sample ages. Several sequences were identified in the library for bacteria associated with disease in humans and poultry such as clostridia, Campylobacter and staphylococci. However, the sequences of bacterial populations varied significantly by age of the birds. There was a unique community structure with the sequences homologous to culturable bacteria such as L. delbrueckii, C. perfringens and Campylobacter coli at 3 days age. From 7 days of age to 21 days, a similar community structure was maintained with dominant sequences related to L. acidophilus, Enterococcus and Streptococcus. To some extent the bacterial community at 49 days of age was similar to those at age 28 with the abundant sequences homologous to L. crispatus, but it was significantly different from those sequence from the other ages. The role of those bacteria nutrient acquisition, intestinal heath and growth promotion remains to be defined.

It has long been known that densely colonized intestinal bacteria play an important role in the health and performance through its effect on gut morphology, nutrition, and pathogenesis of intestinal disease and immune response of animal. Bacteriological changes were found to occur in the intestine of young chickens after they were infected with sporulated oocysts of Eimeria tenella, a parasite of chickens (Kimura, N., F. et al. 1976, Poultry Sci. 55:1375-1383). Intestinal bacteria are primarily responsible for degrading the copious amounts of mucus produced by goblet cells in the intestinal mucosa (Falk, 2000, supra). The microflora is also believed to protect against colonization of the intestines by pathogens and to stimulate the immune response (Mead, G. C. 2000, Vet. J. 159:111-123).

Our present work used molecular techniques to identify the bacterial composition and to determine community succession in the ileum of chickens fed a corn-soy diet lacking coccidiostats and growth-promoting antibiotics. These findings are used to achieve present day growth rate and feed efficiency, without use of antibiotics, by manipulation of the intestinal flora. It is also used to predict intestinal disease prior to the clinical manifestation of symptoms and to prevent colonization of pathogens, such as C. perfringens, Salmonella spp. or Campylobacter spp.

Monoclonal or polyclonal antibodies, preferably monoclonal, specifically reacting with a polypeptide or protein of interest may be made by methods known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York; and Ausubel et al. (1993) Current Protocols in Molecular Biology, Wiley Interscience, New York, N.Y.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, New York; and Ausubel et al. (1993) Current Protocols in Molecular Biology, Greene/Wiley, New York, N.Y. Abbreviations and nomenclature, where employed, are deemed standard in the field and commonly used in professional journals such as those cited herein.

Each reference cited in the present application is incorporated by reference herein to the extent that there is no inconsistency with the present disclosure.

The following examples are provided for illustrative purposes, and are not intended to limit the scope of the invention as claimed herein. Any variations in the exemplified articles which occur to the skilled artisan are intended to fall within the scope of the present invention.

### EXAMPLES

### Example 1

### Sampling

Sixty day-of-hatch commercial leghorn-hybrid broiler chicks, placed on sawdust bedding, were used as the source of bacteria for DNA extractions. Chicks were fed ad libitum commercial corn-soy diet that did not contain growth-promoting antibiotics or coccidiostats. Ten chicks were sacrificed at 3 and 7 days of age, and then the ileal contents were removed and pooled. At 14, 21, 28, and 49 days of age, 5 chicks per age were sacrificed and their ileal contents pooled. The ileum was cut aseptically, and contents were removed to 3 ml tubes containing brain heart infusion broth (BHIB) kept on ice, and processed for bacterial recovery. The contents from the individual birds were pooled to provide a composite sample prior to bacterial fraction recovery, cell lysis, and DNA isolation.

### Example 2

### Recovery of Bacteria, Cell Lysis and DNA Isolation

The bacterial fraction was recovered from the ileum contents through multiple rounds of dilution, high speed centrifugation, and washing with PBS as described previously (Apajalahti et al. 1998, supra). The bacteria were pelleted by a high-speed centrifugation (3,650×g for 15 min.), re-suspended in superbroth (Provence, D. L., and R. Curtiss III, 1994, “Gene transfer in gram-negative bacteria,” pp. 317-347. In P. Gerhardt, Ed., Methods in General and Molecular Bacteriology, ASM Press, Washington D.C.) with 15% glycerol and stored at −80° C. Bacterial cells were lysed using the beads and solution 1 and IRS of Mo Bio kit (Mo Bio Laboratories Inc., Carlsbad, Calif.) by beating at 6000 rpm for 20 min. Genomic DNA was extracted as follows: lysed cells were treated with SDS (0.5%, final concentration), and proteinase K (0.1 mg ml⁻¹, final concentration) and incubated at 37° C. for 30 min. The sample was extracted twice with an equal volume of phenol-chloroform-isoamyl alcohol (PCI, 25:24:1) and once with chloroform-isoamyl alcohol (Cl, 24:1). DNA was isolated with a propanol precipitation. DNA concentration was measured using a Beckman DU640 spectrophotometer (Beckman Instruments Inc., Fullerton, Calif.).

### Example 3

### PCR for Construction of 16S rDNA Clone Libraries

For construction of the 16S rRNA gene clone libraries, three sets of primers, which target the domain Bacteria were used (Hicks et al. 1992). These were (1) 8F, (5′-AGA GTT TGA TCC TGG CTC AG-3′)/1492R (5′-TAC GGY TAC CTT GTT ACG ACT T-3′); SEQ ID NO:12 and SEQ ID NO:13, respectively, (2) 8F/1522R (MG GAG GTGATC CAN CCR CA) and (3) 8F/926R (ACC GCT TGT GCG GGC CC) SEQ ID NO:14 and SEQ ID NO:15, respectively. Y represents C or T, R A or G, and N is A or G or C or T. Primer 1492R contains a single degeneracy, which is between T and C at position 1497 (E. coli numbering). The first two primer sets are frequently used in molecular diversity studies because they result in a nearly full-length 16S rDNA product and are considered universal for the domain Bacteria, and for the prokaryotes (domains Archaea and Bacteria, respectively) (Lane, D. J. 1991, 16S/23S rRNA sequencing, p115-175. In E. Stackebrandt and M. Goodfellow (ed), Nucleic Acid Techniques in Bacterial Systematics, Wiley & Sons, Chichester, United Kingdom). Primer set 3 was used to minimize the effect of template concentration on PCR bias. Final reaction conditions were template DNA 25 ng/μl and 100 ng/ml in the tubes with primer set 3 and 25 ng/ml in the tubes with other primer sets, 1 μl AmpliTaq Goldreaction buffer, 2.0 mM MgCl₂, 0.2 mM dNTP, 1 μM of each primer and 0.05 U of Taq DNA polymerase (AmpliTaq Gold; Perkin-Elmer Corporation, Foster City, Calif. or Roche Diagnostics Corporation, Indianapolis, Ind.) in a final reaction volume of 25 μl. Initial DNA denaturation and enzyme activation steps were performed at 94° C. for 2 min in a PTC200 thermocycler (MJ Research, Inc., Watertown, Mass.), followed by 10-20 cycles, desirably 18, of denaturation at 94° C. for 1 min, annealing at 54° C. (primer set 1), 48° C. (primer set 2) and 58[ ]C (primer set 3) respectively for 30 sec, and elongation at 72° C. for 1 min, which was followed by a final elongation at 72° C. for 10 min. PCR was performed three times for the three reactions to minimize the risk of certain 16S rDNA types being preferentially amplified (Wilson, K. H., and R. B. Blitchington, 1996, Appl. Environ. Microbiol. 62:2273-2278) and to increase the DNA yield. Amplified PCR products were purified with the Wizard PCR product purification kit (Promega, Madison Wis.). Five PCR reaction mixtures were pooled together.

Lu et al. (2003) Appl. Environ. Microbiol. 69:901-908 discloses oligonucleotides useful for PCR amplification-based detection of potentially pathogenic bacteria including Salmonella species, E. coli O157, Staphylococcus aureus, Campylobacter, Yersinia, Listeria and C. perfringens.

In some experiments, the PCR products were loaded onto a gel from which bands were cut and eluted in 35 μl of sterile filtered distilled water using a QIAquick gel extraction kit (Qiagen, Chatsworth, Calif.). The concentrations of the fluorescently labeled PCR products were measured on a spectrophotometer (DU Series 500, Beckman, Fullerton, Calif.). About 100 ng of purified PCR products was digested in a 10 μl volume for 4 hours at 37 C with 10 U of HaeIII (isoschizomer BsuRI; Fermentas, MBI). Restriction digests were desalted with the QIAquick Nucleotide Removal Kit (Qiagen). The fluorescently labeled terminal restriction fragments (T-RFs) were analyzed by electrophoresis on an automatic sequence analyzer (ABI PRISM 310 DNA Sequencer; PE Biosystems, Foster City, Calif.) in GeneScan mode. Aliquots (2 ul) of T-RFs were mixed with 2 μl of deionized formamide, 0.5 μl of DNA fragment length size standard GS-500 (PE Biosystems). The T-RF mixture was denatured at 94° C. for 5 min and immediately chilled on ice prior to electrophoresis. After electrophoresis, the lengths of fluorescently labeled T-RFs were determined by comparison with internal standards by using GeneScan software (ABI). For each sample, peaks over a threshold of 50 units above background fluorescence were analyzed by manually aligning fragments to the size standard. To avoid detection of primers and uncertainties of size determination, terminal fragments smaller than 35 bp and larger than 525 bp were excluded from the analysis. Reproducibility of patterns was confirmed for repeated T-RFLP analysis of 16S gene amplification using the same DNA extracts from pooled samples.

The purified products were ligated into pGEM-T Easy (Promega, Madison, Wis.). Ligation was done at 4° C. overnight followed by transformation into competent E. coli JM109 cells by heat shock (45 sec at 42° C.). We screened the clones for a complementation of β-galactosidase by using X-Gal (5-bromo-4-chloro-3-indolyl-β-D-galactopyranoside) and IPTG (isopropyl-β-D-thiogalactopyranoside). For T-RFLP analyses, the 8F primer was labeled with 5′-FAM (carboxyfluorescein-N-hydroxysuccinimide ester-dimethyl sulfoxide).

### Example 4

### Plasmid Extraction and Sequencing

DNA preparations for sequencing were made with the QIAprep spin plasmid kit (Qiagen, Valencia, Calif.) as specified by the manufacturer. Plasmids were eluted with 50 ml water, and the products were stored at −70° C. Sequencing reactions were performed with a PE-ABI Big Dye Terminator Cycle Sequencing Kit (Applied Biosystems, Foster City, Calif.) as described by the manufacturer, and electrophoresis and readout were done with an ABI PRISM7 3700 DNA Analyzer (Applied Biosystems). Primers T7 and SP6 were used in the sequencing reactions to sequence both strands of each PCR product.

### Example 5

### Analysis of DNA Sequences

Resulting DNA sequences were edited to exclude primer binding sites and ambiguous bases and assembled into contiguous sequences (570-650 bp) using the Sequencher program, version 4.10 (Gene Code Corp., Ann Arbor, Mich.). The programs FASTA (Pearson W. R. 1990 Methods in Enzymology. 183:63-98) and BLAST (Altschul et al. 1997 Nucleic Acids Res. 25:3389402) were used to search GenBank for homologue of contiguous sequences. Chimeric sequences were detected as described (Suau, A. et al. 1999, Appl. Environ. Microbiol. 65:47994807). The estimate of sample size and coverage were conducted according to the formula for coverage as described (Good, I. J. 953, Biometrika, 40:237-264) and applied in quantitative comparisons of 16S rRNA gene sequence libraries by Singleton, D. R. et al. 2001 Appl. Environ. Microbiol. 67:4374-4367). The same definition for the variables in the formula Cx=1−(Nx/n) as in Singleton et al. (2001), was used, i.e., where Cx is the “homologous” coverage of sample X; Nx is the number of unique sequences and n is the total number of sequences in the sample. We used a level of >98 of homology as a criterion by which relatedness was considered by McCaig et al. (1999) (McCaig, A. E. et al. 1999, Appl. Environ. Microbiol. 65:1721-1730) and Suau et el. (1999) supra, respectively. Sequencher were used for all the sequences (430 B 480 bp) from the two primer sets with the same forward primer (8F) to analyze homologous nucleotides. Nine of triplicate sequence samples were randomly taken from 614 consistent sequences to analyze nm and estimate Cs. The differences of 16S rRNA gene sequence libraries between different age samples were estimated using the methods described by Singleton et al. (2001) supra. Representative sequences are available on The National Center for Biotechnological Information website under Accession Nos. AY080963 to AY080994. Printouts of these sequences are included herein below, following the claims.

### Example 6

### Semi-Quantitative Tests of Ratios of Bacterial Template to PCR Product

To evaluate the quantitative consistency of the PCR amplification, we evaluated whether the number of cloned 16S rDNA sequences correlated to the ratio of bacterial genomic DNA template. Bacterial strains Clostridium perfringens ATCC 13124, L. acidophilus ATCC 33199, Bacteroides fragilis ATCC 23745 and Enterococcus faecium ATCC 19434 are available from the American Type Culture Collection (Manassas, Va.) and are grown in the broth media provided with the strains. The DNA extraction was described above. Template ratios for PCR were set as 1:1, 4:1, 16:1 in a total 2.5 ng μl⁻¹ of L. acidophilus to E. faecium, C. perfringens and B. fragilis respectively. Three separate PCR reactions, for each ratio, were performed using the primer sets 8F/1492R following the PCR conditions as described (Lu et al. (2003) Appl. Environ. Microbiol. 69:6816-6824). The 16S rDNA amplicons were then purified and cloned as described by Lu et al. (2003). In order to identify the ratio of 16S rDNA clones, 30 colonies in each plate (2 plates for each PCR reaction) were randomly picked and the identity of the 16S rDNA clone determined by PCR. Species specific primers for L. acidophilus, 5′-CATCCAGTGCAAACCTAAGAG-3′,5′-GATCCGCTTGCCTTCGCA-3′ (SEQ ID NO:16 and NO: 17, respectively) (Wang et al. 1996), Clostridium perfringens, 5′-AAAGGAAGATTAATACCTCATAA-3′,5′-TAAGTTTGGCTCCACCTCGCG-3′ (SEQ ID NO:18 and NO:19, respectively) (Franks et al. 1998), Bacteroides fragilis, 8F and 5′-CCAATGTGGGGGACCTT-3′ (SEQ ID NO:20), and Enterococcus faecium, 5′-GGAAACAGGTGCTAATACCG-3′,5′-GGTTAGATACCGTCAAGGG-3′ (SEQ ID NO:21 and NO:22, respectively). The ratios of resulting clones were determined in six separate experiments in order to evaluate the limitations of quantitative 16S rDNA PCR.

### Example 7

### Statistical Analysis of T-RFLPs

The information index (Shannon and Weaver (1963) “The Mathematical Theory of Communication,” p. 117, University of Illinois Press, Urbana, Ill.) was used to initially evaluate the diversity of the microbial communities.

$${H}_{1} = - {\sum }_{i = 1}^{A} {A}^{{{log}_{2}}_{A}}$$

Where n is possible categories in a data set and that their proportions are pᵢ, . . . , pₙ. The H values are the measure of diversity for this system. To characterize the communities by the numbers of peaks and the area of the peaks, the relative abundance of T-RFs within the sections was determined by calculating the ratio between the areas of each peak and the total areas of all peaks within one sample. Ratioswere converted to percentages.

Gene-specific T-RFLPs from sections within and between cores were compared by correspondence analysis (proc corresp, SAS 8.20) of combined results from three different cleavages using the procedure CORRESPONDENCE from the SAS statistical package (version 6.12; SAS Institute, Cary, N.C.) by considering numbers of peaks and peak heights. The diversity indices were analyzed statistically to determine differences between the control and treatments. PROC GLM Models with t-test was used in SAS (version 8.20, TS2M0).

Where t=1, . . . , I, j=1, . . . , J, and k=1, . . . , K, but represent it in the form
Xᵢⱼₖ=αᵢ+βⱼ+γᵢⱼ+εᵢⱼₖ,
where γᵢⱼ is the interaction of factors A—diet and B—age.

The relevant null hypotheses are
H_{oAB: γ}ᵢⱼ=0, for all i,jH_{oA}: αᵢ=0, i=1, . . . , I, H_{oB}: βⱼ=0, j=1, . . . , J.
   and are tested by their respective F values.

Probability test between the sequences of control group and the treatment with growth promotants and Correlation test between the frequency of sequences and the fluorescent density of peaks were conducted. Representative clone sequences were deposited in GenBank with Accession Numbers AY237182 to AY237208; these are incorporated by reference herein.

### Example 8

### Analyzing Antibiotic-Fed Chickens to Identify Probiotics

Reliable microflora modification approaches, such as probiotic dietary supplements that replace growth-promoting antibiotics in chickens, are developed by characterizing the true composition of the intestinal microflora with different growth-promoting antibiotics.

The bacterial composition is determined, as outlined in the previous examples, by PCR amplification using universal bacterial 16S primers; cloning the PCR products; DNA sequencing the individual clones; and comparing the sequence to known taxonomic groups for identity.

Chickens were fed monensin and compared to a control group fed the same feed without the antibiotic. The microflora in the cecum and ileum were analyzed (see Tables 6-1 to 6-7). We found that monensin reduced the overall numbers of Lactobacillus sequences while the clostridial sequences increased (Table 6-6). Specifically, C. irregularis, C. lituseburense and C. disporicum and the segmented filamentous bacteria comprised a major portion of the bacterial flora of the ileum replacing the lactobacilli (Tables 6-6 and 6-7). These bacteria exclude harmful bacteria, such as C. perfringens, and are responsible for the prevention of enteritis in chicks fed AGPs. Furthermore, this shift in the clostridial population of the ileum is responsible for the growth-promoting qualities.

Therefore, direct feeding of these beneficial species of clostridia can replace the need for using AGPs while maintaining the same beneficial effects, including disease prevention and growth promotion. In addition, measuring the levels of these species serve as an indicator of intestinal microbial health and as a screen for useful prebiotics to promote intestinal health.

### CONCLUSION

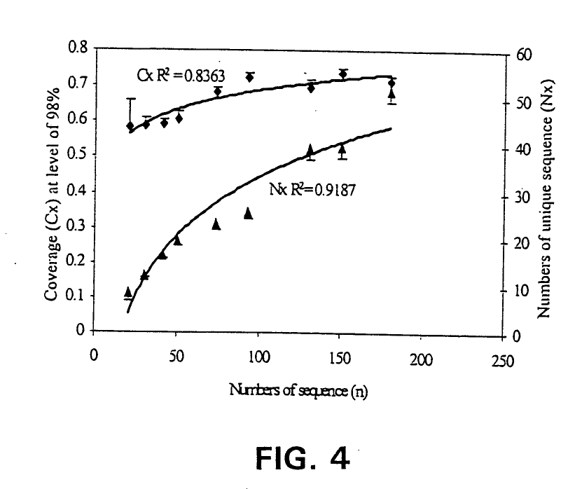

Among 614 sequences analyzed, there were 78 unique sequences at the level of 98% identity. The coverage calculated for the total sequences was 87.79 at the level of 98%. FIG. 4 shows that when sample size n attained is about 130, the curve of both coverage Cs and unique sequences at the level of 98% tended to increase slowly, indicating that minimum sample size for this study could be about 130 sequences which covers about 70% of 98% homologous sequences. Therefore, the total 614 sequences analyzed in this study should be large enough to represent the majority composition of the community in chicken ileum.

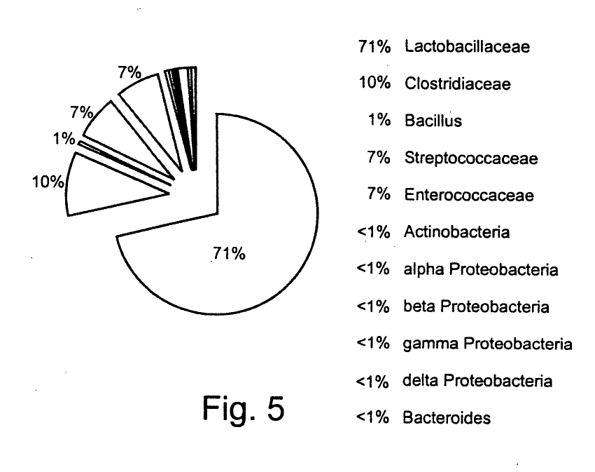

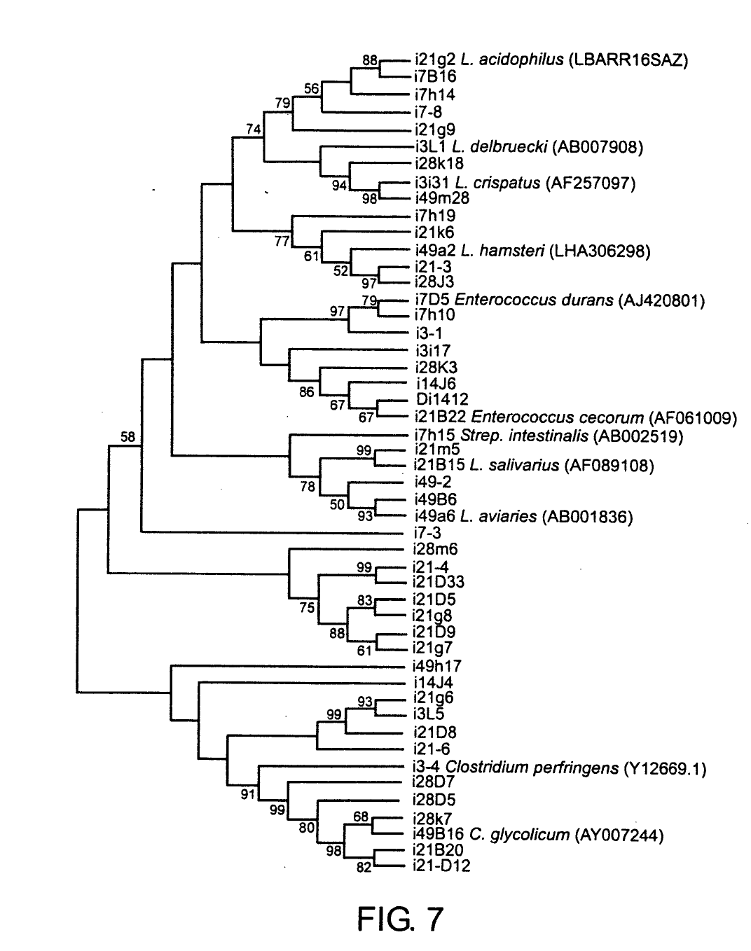

From the analysis of a total of 1230 clones isolated from the 16S rDNA libraries of bacteria collected from broiler litter, we identified four major phyla. These phyla included low and high G+C gram-positives, proteobacteria and the Cytophaga/Flexibacter/Bacteroides (CFB) group (Table 3 and FIG. 5). Eleven families or groups and sixteen genera were identified among the 16S rDNA sequences analyzed. The broiler litter bacterial microbiota consisted predominantly of low G+C gram-positive bacteria, whose representative distinct sequences were shown in FIG. 7, with Lactobacillus accounting for 68.85% of the total 16S rDNA sequences in the libraries. The low G+C gram-positives consisted of five families or groups represented by nine genera. Identification of members of dominant genera Lactobacillus, Enterococcus and Streptococcus were culturable and have been often isolated from normal intestine (Barnes et al. 1972). However, we did not expect to find that Clostridia was a dominant group at age 3 and age 49 in the ileum (Table 3 and FIGS. 5, 6) according to previous studies (Barnes et al. 1972; Salanitro et al. 1978, supra).

TABLE 3
P value distribution of 16S rDNA gene sequence libraries among different
age samples, estimated by pair-wised comparisons based on evolutionary
distance using Jukes-Cantor's method at the level of 95% of coverage.

| Age (days) | 3 | 7 | 14 | 21 | 28 | 49 |
|---|---|---|---|---|---|---|
| 3 | 1 | 0.001 | 0.001 | 0.001 | 0.001 | 0.001 |
| 7 | 0.001 | 1 | 0.048 | 0.041 | 0.001 | 0.001 |
| 14 | 0.001 | 0.937 | 1 | 0.172 | 0.436 | 0.001 |
| 21 | 0.044 | 0.997 | 0.740 | 1 | 0.567 | 0.001 |
| 28 | 0.001 | 0.001 | 0.001 | 0.249 | 1 | 0.028 |
| 49 | 0.001 | 0.001 | 0.001 | 0.001 | 0.124 | 1 |

We compared the sequences for all six ages in a pair-wise manner to determine whether the flora was significantly different. P-value distributions (Table 4) showed that the sequences from age 3 and age 49 were different from all other ages respectively. For other five ages, the sequences from age 7 to age 21 and between age 21 and age 28 have higher similarity. The detailed differences could be easily seen in Table 3 and FIG. 6, in which similar dominant species, L. acidophilus, Clostridium, Streptococcus and Enterococcus, and their abundance were found from age 7 to age 21. These results suggested that the chicken ileum from age 7 to age 21 and between age 21 to age 28 had similar bacterial community structures, but there were very unique community structures at ages 3 and 49. There were obvious successions of dominant species with different ages. The most dominant sequences homologous to Lactobacillus varied from L. delbrueckii at 3 d to L. acidophilus from 7 d to 21 d of age and to L. crispatus from 28 d to 49 d of age. It is interesting to note that the frequencies of the sequences with homology to Clostridium tended to increase from 3 d to 49 d of age. However, C. perfringens specific sequences were prevalent only at 3 d of age.

TABLE 4
rDNA frequencies in ileum of chickens fed corn soy diet without growth-promoting antibiotics or coccidiostats

| | | | 3 day | | 7 day | | 14 day | | 21 day | | 28 day | | 49 day | |
|---|---|---|---|---|---|---|---|---|---|---|---|---|---|---|
| | | | | % of | | % of | | % of | # of | % of | # of | % of | # of | % of |
| Group | | Genus or species | # of seq | seq | # of seq | seq | # of seq | seq | seq | seq | seq | seq | seq | seq |
| Low G + C | Lactobacillaceae | Lactobacillus | 57 | 60.00 | 58 | 64.44 | 65 | 63.73 | 75 | 65.79 | 96 | 87.27 | 69 | 69.70 |
| Gram-positive | | spp. | | | | | | | | | | | | |
| (LGC) | | L. acidophilus, | 7 | | 54 | | 54 | | 57 | | 3 | | | |
| | | L. crispatus, | 4 | | 1 | | 8 | | 3 | | 82 | | 36 | |
| | | L. reuteri | | | | | 3 | | 5 | | 8 | | 1 | |
| | | L. delbrueckii | 40 | | 1 | | | | | | | | | |
| | | Weisella spp. | 6 | | | | | | | | | | | |
| | | L. salivarius | | | | | | | 6 | | 2 | | 28 | |
| | | L. gasseri | | | | | | | 3 | | | | | |
| | Clostridiaceae | Clostridium spp. | 16 | 16.84 | 1 | 1.11 | 7 | 6.86 | 9 | 7.89 | 7 | 6.36 | 19 | 19.19 |
| | | C. perfringens | 15 | | | | | | | | | | | |
| | | Ruminococcus | | | | | | | 3 | | | | | |
| | | Eubacterium spp. | | | | | | | 5 | | | | | |
| | Bacillaceae | Bacillus | | | | | | | | | | | 4 | 4.04 |
| | Staphylococcaceae | Staphylococcus | 2 | 2.11 | | | | | 3 | 2.63 | | | | |
| | Streptococcaceae | Streptococcus | 2 | 2.11 | 16 | 17.78 | 17 | 16.67 | 3 | 2.63 | 1 | 0.91 | | |
| | Enterococcaceae | Enterococcus | 3 | 3.16 | 14 | 15.56 | 13 | 12.75 | 3 | 2.63 | 3 | 2.73 | 2 | 2.02 |
| High G + C | Actinobacteria | Fusobacter | | | | | | | 5 | 4.39 | | | | |
| Gram-positive | | prausnitzii | | | | | | | | | | | | |
| (HGC) | | Bifidobacter | | | 1 | 1.11 | | | | | | | | |
| | Bacteroides | | | | | | | | | | | | | |
| Proteobacteria | Alpha | Ochrobactrum | 1 | 1.05 | | | | | | | | | | |
| (gram- | Beta | Alcaligenes | 4 | 5.26 | | | | | | | | | | |
| negative) | | A. faecalis, | 1 | | | | | | | | | | | |
| | Epsilon | Campylobacter | 5 | 5.26 | | | | | | | | | | |
| | Delta | E. coli | 1 | 2.11 | | | | | | | | | | |
| | | Salmonella | 1 | | | | | | | | | | | |
| | | enterica | | | | | | | | | | | | |
| CFB phylum | Bacteroides | Bacteroides spp. | | | | | | | 3 | 2.6 | | | 1 | 1.01 |

TABLE 5
P value distribution of 16S rDNA gene sequence libraries among different
age samples, estimated by pair-wised comparisons based on evolutionary
distance using Jukes-Cantor's method at the level of 95% of coverage.

| Age in ileum | 3 | 7 | 14 | 21 | 28 | 49 |
|---|---|---|---|---|---|---|
| 3 | 1 | 0.001 | 0.001 | 0.001 | 0.001 | 0.001 |
| 7 | 0.001 | 1 | 0.048 | 0.041 | 0.001 | 0.001 |
| 14 | 0.001 | 0.937 | 1 | 0.172 | 0.436 | 0.001 |
| 21 | 0.044 | 0.997 | 0.740 | 1 | 0.567 | 0.001 |
| 28 | 0.001 | 0.001 | 0.001 | 0.249 | 1 | 0.028 |
| 49 | 0.001 | 0.001 | 0.001 | 0.001 | 0.124 | 1 |
| Age in cecum | 3 | 7 | 14 | 21 | 28 | 49 |
| 3 | 1 | 0.001 | 0.001 | 0.001 | 0.001 | 0.001 |
| 7 | 0.001 | 1 | 0.008 | 0.001 | 0.134 | 0.002 |
| 14 | 0.001 | 0.001 | 1 | 0.231 | 0.743 | 0.293 |
| 21 | 0.001 | 0.001 | 0.10 | 1 | 0.669 | 0.003 |
| 28 | 0.001 | 0.001 | 0.015 | 0.100 | 1 | 0.014 |
| 49 | 0.001 | 0.001 | 0.003 | 0.001 | 0.020 | 1 |

TABLE 6-1
Microbial Composition of the Cecum (%)

| Group | Species | 3 d | 7 d | 14 d | 21 d | 28 d | 49 d |
|---|---|---|---|---|---|---|---|
| LGC | Lactobacillus | 23 | | | 1 | 60 | 80 |
| | Clostridia | 42 | 90 | 83 | 54 | | |
| | C. perfringens | 13 | | | | | |
| | Stept/enteroc | 3 | | | | | |
| HGC | Actinobacterium | | 1 | 9 | 31 | 35 | 9 |
| Proteob | | 16 | | 1 | | | |
| CFB | Bacteroides | | 7 | 5 | 11 | 5 | 6 |

TABLE 6-2
Microbial Composition of the Ileum (%)

| Group | 3 d | 7 d | 14 d | 21 d | 28 d | 49 d |
|---|---|---|---|---|---|---|
| Lactobacillus spp. | 60 | 64 | 64 | 66 | 88 | 70 |
| Clostridiaceae | 17 | 1 | 7 | 9 | 7 | 19 |
| Bacillus | | | | | | 4 |
| Staphylococcus | 2 | | | 3 | | |
| Streptococcus | 2 | 18 | 17 | 3 | 1 | |
| Enterococcus | 3 | 16 | 13 | 3 | 3 | 2 |
| Bifidobacter | | 1 | | | | |
| α-Probeobacteria | 1 | | | | | |
| β-Probeobacteria | 5 | | | | | |
| ε-Probeobacteria | 5 | | | | | |
| δ-Proteobacteria | 2 | | | | | |
| Bacteroides | | | | 3 | | 1 |

TABLE 6-3
Ileum Lactobacillus species
(% of total sequences)

| species | 3 d | 7 d | 14 d | 21 d | 28 d | 49 d |
|---|---|---|---|---|---|---|
| L. acidophilus | 7 | 60 | 53 | 50 | 3 | |
| L. crispatus | 4 | 1 | 8 | 3 | 75 | 36 |
| L. reuteri | | | 3 | 5 | 8 | 1 |
| L. delbrueckii | 42 | 1 | | | | |
| L. salivarius | | | | 6 | 2 | 28 |
| L. gasseri | | | | 3 | | |

TABLE 6-4
Ileum Clostridiaceae
(% of total sequences)

| species | 3 d | 7 d | 14 d | 21 d | 28 d | 49 d |
|---|---|---|---|---|---|---|
| Clostridium spp. | 1 | 1 | 7 | 8 | 7 | 19 |
| C. perfringens | 16 | | | | | |
| Ruminococcus spp. | | | | 3 | | |
| Eubacterium spp. | | | | 5 | | |

TABLE 6-5
Monensin treatment
cecum flora (%)

| species | 28d V | 28d |
|---|---|---|
| Lactobacillus spp. | 6 | 1 |
| Clostridium spp. | 27 | 30 |
| Ruminococcus | 10 | 16 |
| Eubacterium | 12 | 9 |
| Actinobacterium | 43 | 35 |
| Bacteroides | 1 | 5 |

TABLE 6-6
Monensin Effect on the Ileum (%)

| Group | 3 d | 7 d | 14 d | 21 d | 28 d | 49 d |
|---|---|---|---|---|---|---|
| Lactobacillus | 3 [60] | 11 [64] | 21 [64] | 27 [66] | 32 [88] | 45 [70] |
| spp. | | | | | | |
| Clostridiaceae | 2 [17] | 84 [1] | 44 [7] | 47 [9] | 62 [7] | 46 [19] |
| Bacillus | | | | | 1 | 5 [4] |
| Staphylococcus | [2] | | | [3] | | |
| Streptococcus | [2] | [18] | [17] | [3] | 1 [1] | 5 |
| Enterococcus | 9 [3] | 3 [16] | 33 [13] | 11 [3] | 1 [3] | 5 [2] |
| Bifidobacter | | [1] | 2 | | | |
| α- | 1 [1] | | | | | |
| Proteobacteria | | | | | | |
| β- | 3 [5] | | | | | |
| Proteobacteria | | | | | | |
| ε- | [5] | | | | | |
| Proteobacteria | | | | | | |
| δ- | 82 [2] | | | | | |
| Probeobacteria | | | | | | |
| Bacteroides | | | | 2 [3] | | 1 [1] |

[ ] = control

TABLE 6-7
Monensin Effect on the Ileum flora (%)

| species | 3 d | 7 d | 14 d | 21 d | 28 d | 49 d |
|---|---|---|---|---|---|---|
| L. acidophilus | [7] | 4 [57] | [52] | [50] | [3] | |
| L. crispatus | 1 [4] | 4 [1] | 17 [8] | 19 [3] | 17 [75] | 20 [36] |
| L. aviaries | | | | | | 16 |
| L. salivarius | 1 | | | 7 [5] | 2 [2] | 3 [28] |
| L. reuteri | | 3 | [3] | [4] | 11 [7] | 5 [1] |
| Clostridium | 1 [17] | 13 [1] | [7] | [8] | 1 [6] | 1 [19] |
| spp. | | | | | | |
| C. irregularis | | 45 | | 19 | 53 | 3 |
| C. | | 24 | 31 | 22 | 7 | 42 |
| lituseburense | | | | | | |

[ ] = control

TABLE 7
Comparison of ileal bacterial community of chickens fed diets containing feed additives
using 16S rDNA clone libraries (% of seq) and T-RFOP analysis (% of peak areas).

| | 7 days of age | | | | | | | | 28 days of age | | | | | | | |
|---|---|---|---|---|---|---|---|---|---|---|---|---|---|---|---|---|
| | Control | | Probiotic | | AGP | | Monensin | | Control | | Probiotic | | AGP | | Monensin | |
| | % | % | % | % | % | % | % | % | % | % | % | % | % | % | % | % |
| | of | peak | of | peak | of | peak | of | peak | of | peak | of | peak | of | peak | of | peak |
| Group | seq | areas | seq | areas | seq | areas | seq | areas | seq | areas | seq | areas | seq | areas | seq | areas |

| | | | | | | | | | | | | | | | | | |
|---|---|---|---|---|---|---|---|---|---|---|---|---|---|---|---|---|---|
| Low G + C | Lacto- | 64.4 | 55.5 | 28.2 | 26.5 | 47.6 | 13.6 | 12.9 | 7.1 | 87.3 | 85.7 | 3.1 | 22.3 | 23.8 | 40.0 | 31.6 | 27.2 |
| (Gram- | bacillaceae | | | | | | | | | | | | | | | | |
| positive) | | | | | | | | | | | | | | | | | |
| | Clostridiales | 2.2 | 6.1 | 23.9 | 10.8 | 2.4 | 26.8 | 82.4 | 92.89 | 6.4 | 3.5 | 84.4 | 69.4 | 64.3 | 51 | 63.2 | 73.2 |
| | Bacillaceae | | | | | | | | | | | 9.4 | 8.3 | | | 1.05 | |
| | Enterococcus/ | 33.4 | 38.3 | 43.7 | 60.1 | 50 | 59.7 | 2.7 | | 3.64 | 10.8 | | | 4.8 | | 2.1 | |
| | Streptococcus | | | | | | | | | | | | | | | | |
| Proteobacteria | α | | | | | | | 1.35 | | | | | | 2.4 | | 2.1 | |
| (gram- | | | | | | | | | | | | | | | | | |
| negative) | | | | | | | | | | | | | | | | | |
| | β | | | 2.8 | | | | | | | | 3.1 | | 2.4 | | | |
| | γ | | | | | | | | | | | | | 2.4 | | | |
| CFB phylum | Bacteroides | | | 2.6 | | | | | | 1.8 | | | | 9.0 | | | |
| (gram- | | | | | | | | | | | | | | | | | |
| negative) | | | | | | | | | | | | | | | | | |

| | | | | | | | | |
|---|---|---|---|---|---|---|---|---|
| Total Sequences analyzed | 90 | 71 | 42 | 74 | 114 | 32 | 42 | 99 |

We identified several 16S sequences demonstrating homology to bacteria potentially pathogenic for chickens (Table 3). About 15% of the total sequences at 3 days of age had homology to C. perfringens, which is important cause of necrotic enteritis in broilers and which is generally managed or controlled with growth-promoting antibiotics (George et al., 1982 supra; Long, 1973 supra). Also in this sample a few sequences homologous to Alcaligenes faecalis, Campylobacter coli, and E. coli were identified. Clostridium spp. were detected in the ileum flora at all the ages. Clostridia can cause gangrenous dermatitis in poultry (Willoughby, D. H. et al. 1996. J. Vet. Diagn. Invest. 8:259-261). However, segmented filamentous Clostridia are commonly found in healthy animals and we detected sequences homologous to this organism at 14 d of age.

Since we were interested in identifying the effects of feed additives on the small intestinal bacterial community structure, we sought to predict the quantitative relationships between the frequency of certain ribotypes, assessed by relative 16S rDNA clone numbers or relative peak areas in T-RFLP, and the abundance of specific bacterial genera. We were particularly interested in the ratio of Lactobacillus to clostridia because abundant lactobacilli are believed to be an indicator of intestinal health while some Clostridium species are intestinal pathogens. These genera differ greatly in rrn copy number and the difference could skew the Lactobacillus/clostridia ratio resulting from the 16S rDNA quantitation. Therefore, we conducted an experiment to determine the effect of varying template ratio, representing differences in bacterial abundance, on the resulting Lactobacillus 16S rDNA PCR product ratio using genomic DNA extracted from the major genera detected in 16S rDNA clone libraries from the chicken small intestine (Lu 2003). Lactobacillus acidophilus has a genome size of approximately 1.85 megabases (MB) and 5 copies of the rrn operon; Enterococcus faecium, genome size=2.6 MB and mm=6 (Oana 2002); Bacteriodes fragilis, genome size=5.3 MB and rrn=6 (Kuwahara 2002); and Clostridium perfringens, genome size=3.03 MB and rrn=10. The L. acidophilus 16S rDNA PCR product ratio consistently increased with increasing molar amounts of Lactobacillus DNA among the three mixtures of bacterial templates. Although, high variances existed among trials using the same template ratios, our results suggest that experimental variation can be reduced by performing multiple trials using the same template. Differences may also be due to preferential amplification of some rrn types (Farrelly et al. (1995) Appl. Environ. Microbio. 61:2798-2801) and indeed, we found that even small amounts of Bacteroides DNA resulted in a two-fold reduced detection of Lactobacillus. Thus, the abundance of lactobacilli may be underestimated in some experiments where Bacteroides are detected as an abundant group. Therefore, in order to reduce the internal variation associated with using community DNA, we performed 3 replicate PCR reactions for each intestinal community DNA sample that was used in a clone library or evaluated by T-RFLP. In addition, multiple T-RFLP profiles were performed in order to statistically compare the bacterial communities of birds fed different diets. Consequently, the 16S rDNA clone frequencies or T-RF peak areas of abundant species should be related to their molar DNA concentrations in the community DNA samples with the caveat that abundant Bacteroides may reduce the relative abundance of lactobacilli.

In T-RFLP analysis, over 20 unique peaks were detected among the groups based on the T-RF position (fragment size). Our previous study (Lu et al. (2003) supra) had shown that many of the 16S sequences related to Clostridium were unique and would yield unique terminal restriction fragments. Accordingly, in order to identify the bacterial species responsible for a particular terminal restriction fragment, we compiled a data file containing 180 restriction-digestion mapped 16S sequences (starting from position 8: E. coli numbering) retrieved from the clone libraries of each group and clone libraries produced in previous studies (Lu et al (2003) supra; Lu et al. (2003) Appl. Environ. Microbiol. 69:6816-6824). Most of the bacterial species represented by 16S rDNA sequence have their own unique HaeIII cutting sites. Even their relatives whose sequence similarity differences are greater than 2% also have their own unique cutting site enabling identification of most of the different molecular species that exhibit a unique terminal restriction fragment.

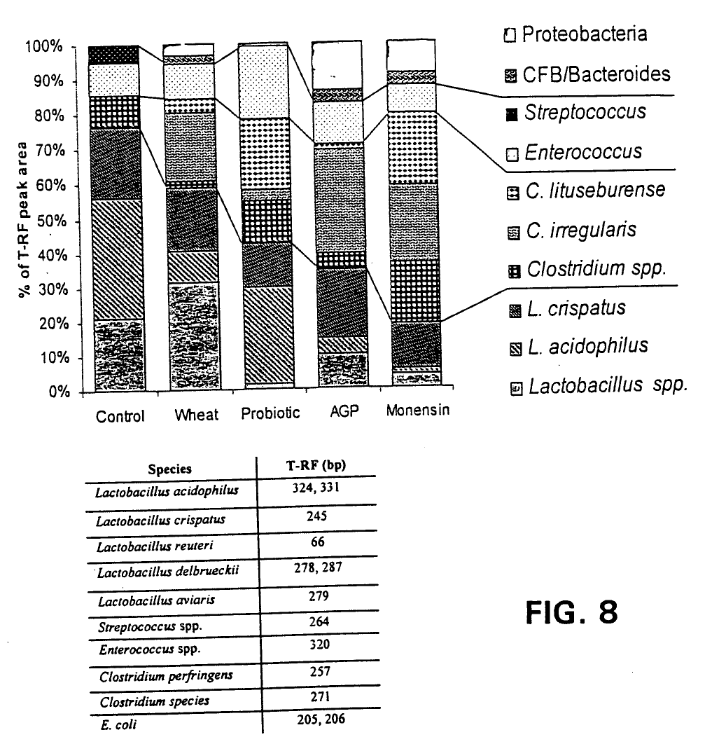

The most abundant bacteria present among the bacterial flora of each group are shown in FIG. 8. The bacterial community was significantly different among control group and some treatment groups. While lactobacilli were prevalent in most groups, the bacterial community of birds fed a corn-soy diet containing monensin consisted of an abundance of clostridia. The control group possessed the highest relative peak areas of Lactobacillus (73.22%) while the monensin group exhibited the lowest (19.25%). However, the monensin and AGP groups also had the highest abundance of Bacteroides; therefore the Lactobacillus abundance was likely underestimated. There was a higher relative abundance of L. acidophilus in control and probiotic groups than the other groups of birds. The relative abundance of L. crispatus and Enterococcus was not greatly different among the groups while the relative abundance of C. irregularis and C. lituseburense, was lowest in the control group and greatest in the AGP, monensin, and wheat group.

In order to better identify and compare the abundant species indicated by the T-RFLP, we produced 16S rDNA clone libraries of the groups at 7 and 28 days of age (Table 3). We confirmed that the most abundant species present in the clone libraries were also represented by the T-RFLP profiles. Detection of the most abundant species was usually consistent between the methods however the relative abundance varied somewhat. Regression analysis, comparing the percent sequence numbers and percent peak areas in each sample (frequency of sequences [%]=−2.5345+1.0347 [peak area %]), confirmed that the methods correlated (N=27, F<0.001, R²=0.728). However there were some differences in the bacterial community structure that appeared to be method-related. For example, T-RFLP was more likely to detect Bacteroides, perhaps because this method employed 3 more cycles of PCR than the clone library method. The clone libraries detected some less abundant members of the community such as Proteobacteria, indicating that the composition of the flora was less likely to be skewed when fewer cycles of PCR are used. With few exceptions, both methods agreed in detecting whether lactobacilli, clostridia, or enterococci were the most abundant group present in a sample. Because of the limitations of these various methods that use ribotype abundance as a semi-quantitative measure of microbial community structure, we adopted a conservative approach that evaluated statistically significant differences among the groups to determine the effects of the various poultry diets on the bacterial flora of the small intestine.

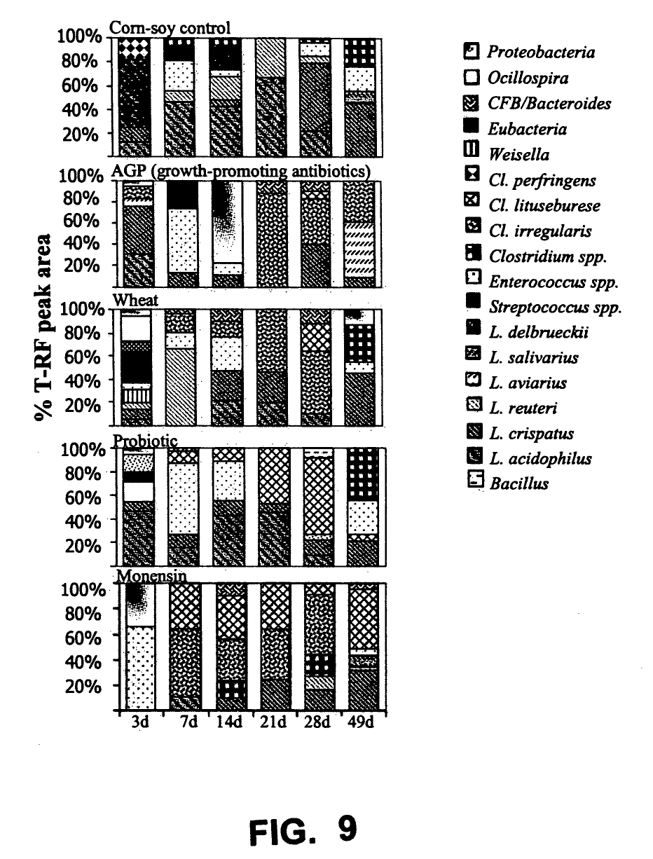

In order to evaluate age-related changes in the composition of the bacterial community, we estimated the abundance of bacterial species among the diet groups (FIG. 9). In addition, the community structure of each sample, represented by T-RF peak numbers and areas, was characterized using the diversity index of Shannon-Weaver (Table 7). Furthermore, correspondence analysis was used to correlate the abundance of bacterial species or genera with certain diet formulations at the different ages. There appeared to be quite different bacterial communities at 3 days of age compared to the other ages and a single factor analysis of variance (df=5, p=0.027) confirmed that the community diversity indices were significantly different. The community diversity indices were highest when the birds were 3 days of age, with the exception of the monensin group where the index was the lowest. A high diversity index suggests evenness in abundance among the species composing the community but does not indicate richness (number of species composing community). The wheat group had the highest diversity index and the highest richness, 8 species comprised the community, while the monensin group had the lowest diversity index and the lowest richness, 2 species. Correspondence analysis showed that the ileal microflora of 3 day-old birds fed monensin were most different from the other groups because of the abundance of Enterococcus hirae and Escherichia coli. In contrast, correspondence analysis showed that the microflora of the probiotic and AGP-fed birds were similar in composition because of the abundance of L. acidophilus and C. irregularis at 3 days of age. The diversity indices of these birds were less than control but the richness was similar. These results suggest that the development of the microflora of very young birds is very susceptible to the effects of various feed additives and diets. The composition of the ileal flora at 7 days was very different from that at 3 days although the birds' diet had not changed. Correspondence analysis showed that the monensin group was again significantly different from the other groups but the birds fed a wheat diet also possessed a unique ileal bacterial flora. LIBSHUFF analysis of the clone libraries showed that the composition of the ileal community of the 7 day old chick was significantly different (p<0.05) among all the groups. Enterococcus was an abundant genus of the community at 7 and 14 days of age in all of the groups except the birds fed the monensin diet. While the diversity indices of most of the groups decreased at this time, the diversity index of the monensin group increased suggesting that the bacterial community complexity increased.

Probiotics are fed to neonatal animals to augment development of a mature intestinal flora. The diversity indices of the probiotic group showed the smallest standard deviation (0.184) of all the groups (0.331-0.465), suggesting that the bacterial flora showed the least amount of instability. At 3 days of age, the ileal bacterial community of the probiotic group was primarily composed of Lactobacillus species and C. irregularis, species that were found to comprise the microflora of older birds in the control group. However, Weisella and Eubacterium were only abundant in 3-day-old birds fed the probiotic, and these bacteria were not commonly detected in older birds in any of the groups. While the probiotic and control groups demonstrated a comparable abundance of lactobacilli and enterococci/streptococci during the first two weeks of age, they exhibited the greatest differences in the types and abundance of clostridia during the rest of the growout period. The correspondence analysis suggested that the ileal community of the probiotic group at 3 and 7 days of age was not greatly different from the control. Therefore, we used LIBSHUFF analysis to determine whether relatedness of the ileal bacterial community of birds in the two groups. The analysis of the clone libraries of the 7-day-old probiotic and control birds showed that they were significantly different (p=0.001) and in fact the clone library of the 7-day-old probiotic group was also significantly different (p=0.001) from the control group at 28 days of age. These data suggest that the use of the probiotic did not result in an ileal bacterial community representative of a mature bird but the probiotic elicited a unique community. Probiotics are usually produced from fecal bacterial communities of adult birds, hence we were interested in whether the ileal community of the 7-day-old probiotic group was similar to the cecal community of the control birds. We used LIBSHUFF to evaluate whether the cloned library of the probiotic group was a subset of the cecal library produced in a previous study (Lu et al. (2003) supra). Both libraries were significantly different from each other (p=0.001) indicating that the probiotic produced a unique ileal community in the treated birds.

The presence of Lactobacillus and Clostridium, the dominant genera of the growing bird (14-28 days of age), was consistent among groups while the presence of other bacteria, such as Enterococcus/Streptococcus, CFB, and proteobacteria, were highly variable. The ileal samples from 14-day-old birds were collected before the grower feed replaced the starter feed. Consequently, 3, 7, and 14-day-old birds in the same groups ate the same feed; 21-28 day old birds were fed grower feed. Therefore, the variation in Enterococcus/Streptococcus, CFB, and proteobacteria abundance was not due to age-related diet changes. Correspondence analysis showed that the ileal flora of birds fed monensin was distinct in its abundance of Clostridium species. This was true at all ages, except 3-days, and despite feed composition changes (starter-grower-finisher) during the growout. In contrast, the ileal flora of the AGP group was highly variable when sampled from 3, 7, and 14-day old birds and exhibited very low diversity indices at all samplings (range 0.357-1.239, mean 0.888±0.331) and low richness (2-5 species). The ileal flora of 14-day-old AGP birds was dominated by an abundance of E. coli while the flora of older birds was composed of primarily of C. irregularis. Antibiotics used as growth promotants are believed to alter the composition, distribution, and metabolism of the intestinal bacteria (Walton, J. R. (1982) J. Vet. Med. Suppl. 33:82). Virginiamycin, for example, has been shown to decrease the levels of cultivable Micrococcaceae, lactobacilli, and Clostridium perfringens from the small intestine of pigs with lesser effects on the cecum (Decuypere, 1973; Vervacke, 1973; Hendericks, 1982). Therefore we investigated whether the microbial community of the birds that were administered AGP was a subset of the control group. LIBSHUFF analysis of the cloned libraries showed that the AGP and controls groups were significantly different (p=0.001) at both 7 and 28 days of age. Therefore the ileal bacterial community of the AGP group was unique.

Although the Clostridiales were abundant in many of the groups, none of the birds demonstrated any gross intestinal pathology. However, the birds fed a wheat diet were visibly smaller than comparison birds during the period of rapid skeletal growth (7-28 days of age) and at the end of the growout, suggesting that either the wheat diet was less digestible or that the microflora did not support comparable feed conversion. Interestingly, the composition of the ileal bacterial flora of the wheat group and the AGP group were very similar during the period of rapid skeletal growth. However, the flora of the AGP group was most dissimilar to the other groups when the birds were 49 days of age (at the end of the growout).

The community structure of each sample, represented by peak numbers and peak areas, was characterized in the diversity index of Shannon-Weaver. The indices ranged from 0.357 (AGP group at 21 d of age) to 1.972 (wheat group at 3 d of age); the indices are shown in Table 5. Comparable mean indices (mean index of all ages) were found among all the groups (1.323-1.193) with the exception of the AGP group (0.888). A two-factor analysis of variance confirmed that there were significant differences between the control group and the AGP group (p=0.0006); in addition, the group receiving monensin (p=0.0847) was significantly different from the control group at the 90% level. There were no significant differences between the control and the other two treatments (wheat, p=0.4003; probiotic, p=0.380). No interaction was detected between age and treatments on the community indices. These results suggest that the ionophore monensin might have enhanced the evenness of bacterial populations (similar abundance) in the microbial community structure, while growth promotants decreased evenness. A high diversity index suggests evenness among the species composing the community but does not indicate richness (number of species composing community). The bacterial community with the highest diversity index, 1.972, was composed of 9 bacterial species while the community with the lowest, 0.357, was only composed of 2 species. Communities with diversity indices of 1.2-1.8 were usually composed of at least 4 species of similar abundance; no index near 1 was composed of fewer than 3 species suggesting that most of the detected bacteria had similar abundance. The relative abundance can be seen in FIG. 9; the ileal communities of chickens at 21 d of age demonstrated consistently low diversity indices, and few abundant species (2-3) suggesting that this period may represent a transitional ileal community.

Although the TRFLP patterns could be directly used to evaluate environmental microbial community as did in previous studies (Liu et al, 1997 and Leser et al. 2000), it is necessary to determine the component and relative quantity of T-RF in order to reveal accurately bacterial community structure of specific samples. Our determination of the T-Rf's component was accurate, because the TRFLP patterns for all the samples were rerun for several times and they were reproducible, the main T-RF peaks were predetermined from the our HaeIII cutting map. Furthermore, some representative samples were cloned and sequenced to confirm the components of their T-RF patterns. The use of the sequence frequency of some bacterial species present in cloning library and the percent T-RF peak height or area as quantitative information to interpret the relative abundance of the bacterial species has been debated, along with the 16S rRNA gene using in the study of microbial community because the bias of in PCR (Farrelly (1995) supra). To minimize the bias of PCR, we amplified 16S rDNA in the conditions of high template concentrations (2.5 ng/μl), fewer cycles (18) and mixing replicate reaction preparations, as known in the art. Prior reports of studies of template-to-product ratios in multitemplate PCR support the validity of quantitative PCR approaches. Using 16S rRNA genes by PCR and detection of PCR products terminal-labeled by FMA and digested with HaeIII showed that the ratios of different PCR products were accurately represented by the ratios of peak areas, although biased. PCR-based TRFLP could reveal the main compositions and relative abundance of environmental bacteria in exert to decrease the 16S PCR biases.

The microbial community may be rather sensitive to diet treatments. Henderick (1982) observed that a change in distribution of the microflora caused by antibiotics, virginiamycin was primarily in the small intestines with lesser effects in the cecum. We conducted the study of effects of different treatments on microbial flora in the ileums of chickens. The bacterial community of the control group in which only corn soy diet was fed showed that lactobacilli were dominant (73.22%), but Clostridium counted for only 8.72%. The previous studies based on the cultures also found that lactobacilli predominate in the small intestine of chickens (Salanitro et al. (1978) supra). With feeding of a corn-based diet, analysis of the ileum of young chicks (14 days) showed that the predominant bacteria were Lactobacillus (33.8-59%), while the other groups, such as Streptococcus, E. coli and eubacteria and clostridium, were a small part, suggesting that the “beneficial” bacteria (Onifade, 1999) which could prevent digestive disorders and/or improve performance in broiler chickens, dominated in the control group. It seems this is a healthy intestinal microflora.

In our study the animals fed the corn-soy diet plus Aviguard showed a significant increase of L. acidophilus, which is believed to have been caused by the Aviguard. This commercial feed is used to establish a “normal gut flora” in chickens and turkeys, according to the producer (Bayer Animal Health). The relative increase in L. acidophilus may reduce the colonization of transient enterobacteria by competitive exclusion (CE). These CE effects include competitive exclusion of pathogens improve digestion and absorption of nutrients and decrease net ammonia production. In the gastrointestinal tract of the broiler chicken, Netherwood et al. (1999) showed that the relative amount of E. faecalis in the total eubacterial population increased in the presence of the non-genetically modified strain and decreased in the presence of the genetically modified probiotics compared with the results obtained with an untreated control group. They suggested that E. faecalis and E. faecium might occupy similar niches or even have a synergistic relationship.

It was obvious that the microbial communities from the ileum of chickens fed with growth promotants (virginiamycin) were significantly different from the control, indicating antibiotics affected the ileum microbial communities. The effect of antibiotics on lactobacilli, especially L. acidophilus, were more significant than other bacteria. Previous culture-based studies also suggested that the antibiotics might damage some bacteria while sparing others (Walton et al. (1982) supra). The effects included significant decreases in Micrococcaceae, lactobacilli, and Clostridium perfringens. These changes in the microflora were accompanied by a 60% reduction in ammonia and a decrease in amine concentration in the small intestines. In an in vitro continuous cultivation system of ileal contents, virginiamycin caused a significant reduction in carbohydrate breakdown. Although the mechanism of growth enhancement by antibiotics is not understood, the beneficial effects are clear. For example, pigs fed virginiamycin (50 ppm) experience a 10% improvement in growth rate and 7% enhancement in feed conversion compared to controls (Hendericks, 1982). It seems that the performance of chicken improved by antibiotics is not through enhancement of ‘beneficial’ bacteria. Rather, antibiotics decreased the lactobacilli according to our results, as had previously been shown.

Another antibiotic, monensin, added to chicken diets, has been used as feed additive in the cattle industry as well. Monensin alters ruminal bacteria by inhibiting gram-positive bacteria, which produce large amounts hydrogen, a precursor of methane, and ammonia (Callaway et al. (1999) Appl. Environ. Microbiol. 65:4753-4759). L. acidophilus may be also sensitive to monensin, but it is interesting to note that C. irregularis was not inhibited in our study. A previous report indicated that, based on 16S rRNA probe hybridization, the relative numbers of Lachnospira multiparus-like organisms decreased about 2-fold with monensin supplementation. Lactobacilli have complex nutritional requirements such as amino acids, peptides, nucleic acid derivatives, vitamins, salts, fatty acid esters, and fermentable carbohydrates for growth. Some of these complex nutrients probably decreased in the small intestine after addition of monensin and antibiotics.

The bacterial community in chickens fed a wheat diet was most different from that of control, indicating basic diet could be very important for a certain bacterial community structure in the chicken intestines. Apajalahti et al. (2001) analysed 144 cecal samples of birds being fed either wheat, or corn or rye. Their results showed that each of the grains favors some bacterial groups in the cecum. It assumed that corn favors low G+C clostridia and campylobacteria, rye stimulates the growth of lactobacilli and enterococci, while wheat favors propionibacteria and bifidobacteria. His results suggested that bacterial communities are significantly correlated with diets, but his analysis based on the G+C proportions by which very different compositional combinations of bacteria might be inferred. Our results showed that corn-soy tended to favor the lactobacilli and wheat favor the clostridia C. lituseburense and C. irregularis.

Thus far we have found that the clostridia C. lituseburense and C. irregularis and their relatives were main components in the treatment groups and their relative abundance vary significantly relative to diets such as wheat and the addition of growth promotants and monensin to corn soy diet, but there have not been evidences to document their correlations to the health and performance of chicken or other poultry. Those bacteria may have commensal host-bacterial relationships in the gut as Hooper and Gordon (2001) proposed, who suggested that these bacteria may directly influence the intestinal epithelium to limit immune activation and to help fortify the epithelial barrier, but they may shift from commensalism toward pathogenicity in certain diseases.

What is claimed is:

1. A probiotic composition comprising viable cells of at least one bacterium selected from the group consisting of Lactobacillus reuteri, Lactobacillus delbreukii, Lactobacillus aviarius, Lactobacillus crispatus, Lactobacillus salivarius, Clostridium irregularis, Clostridium lituseburense and Clostridium disporicum.

2. The composition of claim 1, wherein said composition comprises viable cells of at least two bacteria selected from said group.

3. The composition of claim 1, wherein said composition comprises viable cells of at least three of said group.

4. A method for identifying specific bacteria to be used in a probiotic product, said method comprising the steps of:
(a) comparing gastrointestinal tract bacteria in an antibiotic-fed and a no-antibiotic-fed animal using molecular techniques to identify bacteria present in the antibiotic-fed animal but not in the no-antibiotic fed animal; and
(b) dentifying bacteria of step (a) present in the no-antibiotic-fed and present in lower numbers or absent in an antibiotic-fed animal.

5. The method of claim 4, wherein the bacteria measured include at least one member of the group consisting of Lactobacillus reuteri, Lactobacillus delbreukii, Lactobacillus aviarius, Lactobacillus crispatus, Lactobacillus salivarius, Clostridium irregularis, Clostridium lituseburense and Clostridium disporicum.

6. The method of claim 4, wherein said animal is a mammal, reptile or amphibian.

7. The method of claim 4, wherein said animal is a bird.

8. The method of claim 7, wherein said bird is poultry.

9. The method of claim 8, wherein said poultry is a chicken.

10. The method of claim of claim 4, wherein said bacteria are analyzed in a fecal sample of the animal.

11. The method of claim of claim 10, wherein the animal is a chicken.

12. A method for promoting growth in an animal, said method comprising the step of supplementing animal feed with viable cells of at least one bacteria selected from the group consisting of Clostridium irregularis, Clostridium lituseburense, Clostridium disporicum, Lactobacillus reuteri, Lactobacillus delbreukii, Lactobacillus aviarius, Lactobacillus crispatus, and Lactobacillus salivarius, in an amount sufficient to colonize the animal fed said animal feed, wherein said animal feed does not also comprise an antibiotic.

13. The method of claim 12, wherein the bacteria comprise at least two bacteria of said group.

14. A method for identifying a test composition as a prebiotic for use in an animal feed, said method comprising the step of measuring intestinal microbial levels of clostridia in a gastrointestinal tract of the animal in the presence and absence of a test composition, whereby a test composition is identified as a prebiotic for use in an animal when the level of at least one of C. irregularis, C. lituseburense, C. disporicum, and Lactobacillus reuteri, Lactobacillus delbreukii, Lactobacillus aviarius, Lactobacillus crispatus and Lactobacillus salivarius is greater in a gastrointestinal tract of the animal in the presence than in the absence of said test composition.

15. The method of claim 14, wherein said animal is a mammal, reptile or amphibian.

16. The method of claim 14, wherein said animal is a bird.

17. The method of claim 15, wherein said bird is poultry.

18. The method of claim 17, wherein said poultry is a chicken.

19. The method of claim 14, wherein the level of at least one bacterium selected from the group consisting of C. irregularis, C. lituseburense, C. disporicum, Lactobacillus reuteri, Lactobacillus delbreukii, Lactobacillus aviarius, Lactobacillus crispatus, and Lactobacillus salivarius, is measured in feces of the animal.

20. A method for preventing necrotic enteritis in an animal, said method comprising orally administering an effective amount of viable cells of at least one species selected from the group consisting of C. irregularis, C. lituseburense, C. disporicum, Lactobacillus reuteri, Lactobacillus delbreukii, Lactobacillus aviarius, Lactobacillus crispatus, and Lactobacillus salivarius cells to the animal.

21. The method of claim 20, wherein said animal is a mammal, reptile or amphibian.

22. The method of claim 20, wherein the animal is a bird.

23. The method of claim 22, wherein the bird is poultry.

24. The method of claim 23, wherein the poultry is a chicken.

25. A method for assessing health of an animal, said method comprising the step of measuring C. irregularis, C. lituseburense and C. disporicum, Lactobacillus reuteri, Lactobacillus delbreukii, Lactobacillus aviarius, Lactobacillus crispatus, and Lactobacillus salivarius in a gastrointestinal tract of the animal or in feces of said animal, whereby.

26. The method of claim 25, wherein said animal is a bird.

27. The method of claim 26, wherein said bird is poultry.

28. The method of claim 26, wherein said poultry is a chicken.

29. The method of claim 24, wherein said animal is a mammal, reptile or amphibian.

30. The method of claim 24, wherein the level of at least one of C. irregularis, C. lituseburense and C. disporicum is measured in feces of the animal.

31. The method of claim 24, wherein the level of at least one of Lactobacillus reuteri, Lactobacillus delbreukii, Lactobacillus aviarius, Lactobacillus crispatus, and Lactobacillus salivarius is measured in feces of the animal.


SEQUENCE LISTING (22 sequences)

SEQ ID NO: 1 - DNA, 1568 bp, Lactobacillus acidophilus
  misc_feature (11)..(12): n is a, c, g, or t
agagtttgatnntggctcaggacgaacgctggcggcgtgcctaatacatgcaagtcgagcgagcttgcctagatgattttagtgcttgcactaaatgaaactagatacaagcgagcggcggacgggtgagtaacacgtgggtaacctgcccaagagactgggataacacctggaaacagatgctaataccggataacaacactagacgcatgtctagagtttgaaagatggttctgctatcactcttggatggacctgcggtgcattagctagttggtaaggtaacggcttaccaaggcaatgatgcatagccgagttgagagactgatcggccacattgggactgagacacggcccaaactcctacgggaggcagcagtagggaatcttccacaatggacgaaagtctgatggagcaacgccgcgtgagtgaagaagggtttcggctcgtaaagctctgttggtagtgaagaaagatagaggtagtaactggcctttatttgacggtaattacttagaaagtcacggctaactacgtgccagcagccgcggtaatacgtaggtggcaagcgttgtccggatttattgggcgtaaagcgagtgcaggcggttcaataagtctgatgtgaaacgcttcggctcaaccggagaattgcatcagaaactgttgaacttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatccagtgcaaacctaagagattaggtgttcccttcggggacgctgagacaggtggtgcatggctgtcgtcagctcgtgtcgtgagatgttgggttaagtcccgcaacgagcgcaacccttgtcattagttgccatcattaagttgggcactctaatgagactgccggtgacaaaccggaggaaggtggggatgacgtcaagtcatcatgccccttatgacctgggctacacacgtgctacaatggacggtacaacgagaagcgaacctgcgaaggcaagcggatctcttaaagccgttctcagttcggactgtaggctgcaactcgcctacacgaagctggaatcgctagtaatcgcggatcagcacgccgcggtgaatacgttcccgggccttgtacacaccgcccgtcacaccatgagagtctgtaacacccaaagccggtgggataacctttataggagtcagccgtctaaggtaggacagatgattagggtgaagtcgtaacaaggtagccgtaggagaacctgcggctggatcacctcctttat

SEQ ID NO: 2 - DNA, 1516 bp, Lactobacillus delbrueckii
gacgaacgctggcggcgtgcctaatacatgcaagtcgagcgagctgaattcaaagatyccttcgggrtgatttgttggacgctagcggcggatgggtgagtaacacgtgggcaatctgccctaaagactgggataccacttggaaacaggtgctaataccggataacaacatgaatcgcatgattcaagtttgaaaggcggcgtaagctgtcactttaggatgagcccgcggcgcattagctagttggtggggtaaaggcctaccaaggcaatgatgcgtagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggatagaggcagtaactggtctttatttgacggtaatcaaccagaaagtcacggctaactacgtgccagcagccgcggtaatacgtaggtggcaagcgttgtccggatttattgggcgtaaagcgagcgcaggcggaatgataagtctgatgtgaaagcccacggctcaaccgtggaactgcatcggaaactgtcattcttgagtgcagaagaggagagtggaattccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagcgctaggtgttggggactttccggtcctcagtgccgcagcaaacgcattaagcgctccgcctggggagtacgaccgcaaggttgaaactcaaaggaattgacgggggcccgcacaagcggtggagcatgtggtttaattcgaagcaacgcgaagaaccttaccaggtcttgacatcctgtgctacacctagagataggtggttcccttcggggacgcagagacaggtggtgcatggctgtcgtcagctcgtgtcgtgagatgttgggttaagtcccgcaacgagcgcaacccttgtctttagttgccatcattaagttgggcactctaaagagactgccggtgacaaaccggaggaaggtggggatgacgtcaagtcatcatgccccttatgacctgggctacacacgtgctacaatgggcagtacaacgagaagcgaacccgcgagggtaagcggatctcttaaagctgttctcagttcggactgcaggctgcaactcgcctgcacgaagctggaatcgctagtaatcgcggatcagcacgccgcggtgaatacgttcccgggccttgtacacaccgcccgtcacaccatggaagtctgcaatgcccaaagtcggtgggataacctttataggagtcagccgcctaaggcagggcagatgactggggtgaagtcgtaacaaggtagccgtaggagaacc

SEQ ID NO: 3 - DNA, 1510 bp, Lactobacillus crispatus
ggacgaacgctggcggcgtgcctaatacatgcaagtcgagcgagcagaactaacagatctacttcggtagtgacgtttcggaagcgagcggcggatgggtgagtaacacgtgggtaacctgcccttaagtctgggataccatttggaaacaggtgctaataccggataacaacattgatcgcatgatcgatgcttgaaaggcggcgtaagctgtcgctaaaggatggacccgcggtgcattagctagttggtaaggtaacggcttaccaaggcaacgatgcatagccgagttgagagactgatcggccacattgggactgagacacggcccaaactcctacgggaggcagcagtagggaatcttccacaatgggcgaaagcctgatggagcaacgccgcgtgagtgaagaaggttttcggatcgtaaagctctgttgttggtgaagaaggatagaggtagtaactggcctttatttgacggtaatcaaccagaaagtcacggctaactacgtgccagcagccgcggtaatacgtaggtggcaagcgttgtccggatttattgggcgtaaagcgagcgcaggcggaagaataagtctgatgtgaaagccctcggcttaaccggggaagtgcatcggaaactgtttttcttgagtgcagaagaggagagtggaactccatgtgtagcggtggaatgcgtagatatatggaagaacaccagtggcgaaggcggctctctggtctgcaactgacgctgaggctcgaaagcatgggtagcgaacaggattagataccctggtagtccatgccgtaaacgatgagtgctaagtgttgggaggtttccgcctctcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatctagcgcaattcgtagagatacgaagttcccttcggggacgctaagacaggtggtgcatggctgtcgtcagctcgtgtcgtgagatgttgggttaagtcccgcaacgagcgcaacccttgtcattagttgccagcattaagttgggcactctaatgagactgccggtgacaaaccggaggaaggtggggacgacgtcaagtcatcatgccccttatgacctgggctacacacgtgctacaatgggcagtacaacgagaagcaaacctgcgaaggcaagcgaatctctgaaagctgttctcagttcggactgtaggctgcaactcgcctacacgaagctggaatcgctagtaatcgcggatcagcacgccgcggtgaatacgttcccgggccttgtacacaccgcccgtcacaccatggaagtctgcaatgcccaaagccggtggcctaaccttcgggaaggagccgtctaaggcagggcagatgactggggtgaagtcgtaacaaggtagccgtag

SEQ ID NO: 4 - DNA, 1510 bp, Lactobacillus hamsteri; identical to SEQ ID NO: 3

SEQ ID NO: 5 - DNA, 2898 bp, Enterococcus durans
tttgattatggctcaggacgaacgctggcggcgtgcctaatacatgcaagtcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgaacgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgatttgattatggctcaggacgaacgctggcggcgtgcctaatacatgcaagtcgtacgcttctttttccaccggagcttgctccaccggaaaaagaagagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgaacgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcgaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtga

SEQ ID NO: 6 - DNA, 1509 bp, Enterococcus cecorum
gacgaacgctggcggcgtgcctaatacatgcaagtcgaacgcattttctttcaccgtagcttgctacaccggaagaaaatgagtggcgaacgggtgagtaacacgtgggtaacctgcccatcagcgggggataacacttggaaacaggtgctaataccgcataattccatttaccgcatggtagatggatgaaaggcgcttttgcgtcactgatggatggacccgcggtgcattagctagttggtggggtaacggcctaccaaggctgcgatgcatagccgacctgagagggtgatcggccacactgggactgagacacggcccagactcctacgggaggcagcagtagggaatcttcggcaatggacgcaagtctgaccgagcaacgccgcgtgagtgaagaaggttttcggatcgtaaaactctgttgttagagaagaacaaggatgagagtggaaagttcatcccttgacggtatctaaccagaaagccacggctaactacgtgccagcagccgcggtaatacgtaggtggcaagcgttgtccggatttattgggcgtaaagcgagcgcaggcggtcttttaagtctgatgtgaaagcccccggcttaaccggggagggtcattggaaactgggagacttgagtgcagaagaggaaagcggaattccatgtgtagcggtgaaatgcgtagatatatggaggaacaccagtggcgaaggcggctttctggtctgtaactgacgctgaggctcgaaagcgtggggagcaaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacggggacccgcacaagcggtggagcatgtggtttaattcgaagcaacgcgaagaaccttaccaggtcttgacatcctttgaccatcctagagataggattttcccttcggggacaaagtgacaggtggtgcatggttgtcgtcagctcgtgtcgtgagatgttgggttaagtcccgcaacgagcgcaacccttattgttagttgccatcattcagttgggcactctagcgagactgccgcagacaatgcggaggaaggtggggatgacgtcaaatcatcatgccccttatgacctgggctacacacgtgctacaatggagagtacaacgagtcgcaaagccgcgaggctaagccaatctcttaaagctcttctcagttcggattgtaggctgcaactcgcctacatgaagccggaatcgctagtaatcgcggatcagcacgccgcggtgaatacgttcccgggtcttgtacacaccgcccgtcacaccacgagagtttgtaacacccaaagccggtgcggtaaccgcaaggagccagccgtctaaggtgggatagatgattggggtgaagtcgtaacaaggtagccgtatcggaa

SEQ ID NO: 7 - DNA, 1491 bp, Streptococcus intestinalis
tcctgctcaggcgagccttgcgcgtgcctatacatgcaactacacgcctgaaagagggagcctgcctccctttggatgagttgcgaacgggtgagtaacgcgtaggtaacctgccttgtagcgggggataactattgggaacgatagctaataccgcataagagctttgacacatgttagaagcttgaaaagatgcaattgcatcactacgagatggacctgcgttgtattagctagtaggtagggtaacggcttacctaggcgacgatacatagccgacctgagagggtgatcggccacactgggactgagacgcggcccagactcctacggaagccagcagtagggaatcttcggcaatgggggcaaccctgaccgagcaacgccgcgtgagtgaagaaggttttcggatcgtaaagctctgttgtaagagaagaacgtgtgtgagagtggaaagttcacacagtgacggtaacttaccagaaagggacggctaactacgtgccagcagccgcggtaatacgtaggtcccgagcgttgtccggatttattgggcgtaaagcgagcgcaggcggtttaataagtctgaagttaaaggcagtggcttaaccattgttcgctttggaaactgttaaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccagtgaccgctctagagatagagtttttcttcggaacactggtgacaggtggtgcatggttgtcgtcagctcgtgtcgtgagtagttgggttaagtcccgcaacgagcgcaacccctattgttagttgccatcattcagttgggcactctagcgagactgccggtaataaaccggaggaaggtggggatgacgtcaaatcatcatgccccttatgacctgggctacacacgtgctacaatggttggtacaacgagtcgcaagtcggtgacggcaagcaaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccgtttaggagccagccgcctaaggttggatagatgattggggtgaagtcgtaacaag

SEQ ID NO: 8 - DNA, 1587 bp, Lactobacillus salivarius
ccgaattcgtcgacaacagagtttgatcctggctcaggacgaacgctggcggcgtgcctaatacatgcaagtcgaacgaaactttcttacaccgaatgcttgcrttcatcgtaagaagttgagtggcggacgggtgagtaacacgtgggtaacctgcctaaaagaaggggataacacttggaaacaggtgctaataccgtatatctctaaggatcgcatgatccttagatgaaagatggttctgctatcgcttttagatggacccgcggcgtattaactagttggtggggtaacggcctaccaaggtgatgatacgtagccgaactgagaggttgatcggccacattgggactgagacacggtccaaactcctacgggaggcagcagtagggaatcttccacaatggacgcaagtctgatggagcaacgccgcgtgagtgaagaaggtcttcggatcgtaaaactctgttgttagagaagaacacgagtgagagtaactgttcattcgatgacggtatctaaccagcaagtcacggctaactacgtgccagcagccgcggtaatacgtaggtggcaagcgttgtccggatttattgggcgtaaagggaacgcaggcggtcttttaagtctgatgtgaaagccttcggcttaaccggagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgaatgctaggtgttggagggtttccgcccttcagtgccgcagctaacgcaataagcattccgcctggggagtacgaccgcaaggttgaaactcaaaggaattgacgggggcccgcacaagcggtggagcatgtggtttaattcgaagcaacgcgaagaaccttaccaggtcttgacatcctttgaccacctaagagattaggctttcccttcggggacaaagtgacaggtggtgcatggctgtcgtcagctcgtgtcgtgagatgttgggttaagtcccgcaacgagcgcaacccttgttgtcagttgccagcattaagttgggcactctggcgagactgccggtgacaaaccggaggaaggtggggacgacgtcaagtcatcatgccccttatgacctgggctacacacgtgctacaatggacggtacaacgagtcgcaagaccgcgaggtttagctaatctcttaaagccgttctcagttcggattgtaggctgcaactcgcctacatgaagtcggaatcgctagtaatcgcgaatcagcatgtcgcggtgaatacgttcccgggccttgtacacaccgcccgtcacaccatgagagtttgtaacacccaaagccggtggggtaaccgcaaggagccagccgtctaaggtgggacagatgattggggtgaagtcgtaacaaggtagccgtaggagaacctgcggctggatcacctccttaagcttggatcccggg

SEQ ID NO: 9 - DNA, 1510 bp, Lactobacillus aviarius
agagtttgatcctggctcaggacgaacgctggcggcgtgcctaatacatgcaagtcgaacgagaatttcttacaccgagtgcttgcactctaccgtaagaaattcgagtggcggacgggtgagtaacacgtgggtaacctgcccaaaagaaggggataacatttggaaacaaatgctaataccgtataaccatgatgaccgcatggtcattatgtaaaaggtggttttgctatcgcttttggatggacccgcggcgtattaactagttggtagggtaacggcctaccaaggtgatgatacgtagccgagttgagagactgatcggccacaatgggactgagacacggcccatactcctacgggaggcagcagtagggaatcttccacaatgggcgcaagcctgatggagcaacgccgcgtgaatgaagaaggtcttcggatcgtaaaattctgttgttagagaagaatatgagtaatagtaactgattattcactgacggtatctaaccagcaagtcacggctaactacgtgccagcagccgcggtaatacgtaggtggcaagcgttgtccggatttattgggcgtaaagggaacgcaggcggttttttaagtctgatgtgaaagccttcggcttaaccggagatgtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcatgggtagcgaacaggattagataccctggtagtccatgccgtaaacgatgaatgctaggtgttggagggtttccgcccttcagtgccgcagctaacgcaataagcattccgcctggggagtacgaccgcaaggttgaaactcaaaggaattgacgggggcccgcacaagcggtggagcatgtggtttaattcgaagcaacgcgaagaaccttaccaggtcttgacatcttttgaccacctaagagattaggttttcccttcggggacaaaatgacaggtggtgcatggctgtcgtcagctcgtgtcgtgagatgttgggttaagtcccgcaacgagcgcaacccttgttgtcagttgccatcattcagttgggcactctggcgagactgccggtgacaaaccggaggaaggtggggatgacgtcaagtcatcatgccccttatgacctgggctacacacgtgctacaatggacgatacaacgagtcgcgaaaccgcgaggttaagctaatctcttaaagtcgttctcagttcggattgcaggctgcaactcgcctgcatgaagtcggaatcgctagtaatcgcggatcagcatgccgcggtgaatacgttcccgggccttgtacacaccgcccgtcacaccatgagagtttgtaacacccaaagccggtggagtaaccatttggagctagccgtctaaggtgggactgatgatgagggtgaagt

SEQ ID NO: 10 - DNA, 1504 bp, Clostridium perfringens
gagagtttgatcctggctcaggatgaacgctggcggcgtgcttaacacatgcaagtcgagcgatgaagtttccttcgggaaacggattagcggcggacgggtgagtaacacgtgggtaacctgcctcatagagtggaatagccttccgaaaggaagattaataccgcataatgttgaaagatggcatcatcattcaaccaaaggagcaatccgctatgagatggacccgcggcgcattagctagttggtggggtaacggcctaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgaaggttttcggatcgtaaagctctgtctttggggaagataatgacggtacccaaggaggaagccacggctaactacgtgccagcagccgcggtaatacgtaggtggcgagcgttatccggatttactgggcgtaaagggagcgtaggcggatgattaagtgggatgtgaaatacccgggctcaacttgggtgctgcattccaaactggttatctagagtgcaggagaggagagtggaattcctagtgtagcggtgaaatgcgtagagattaggaagaacaccagtggcgaaggcgactctctggactgtaactgacgctgaggctcgaaagcgtggggagcaaacaggattagataccctggtagtccacgccgtaaacgatgaatactaggtgtgggggtttcaacacctccgtgccgccgctaacgcattaagtattccgcctggggagtacggtcgcaagattaaaactcaaaggaattgacggggacccgcacaagtagcggagcatgtggtttaattcgaagcaacgcgaagaaccttacctacacttgacatcccttgcattactcttaatcgaggaaatccttcggggacaaggtgacaggtggtgcatggttgtcgtcagctcgtgtcgtgagatgttgggttaagtcccgcaacgagcgcaacccttgtcgttagttactaccattaagttgaggactctagcgagactgcctgggttaaccaggaggaaggtggggatgacgtcaaatcatcatgccccttatgtgtagggctacacacgtgctacaatggctggtacagagagatgcaataccgcgaggtggagccaaacttaaaaaccagtctcagttcggattgtaggctgaaactcgcctacatgaagctggagttactagtaatcgcgaatcagaatgtcgcggtgaatacgttcccgggtcttgtacacaccgcccgtcacaccatgagagttggcaatacccgaagtccgtgagctaaccgcaaggaggcagcggccgaagtagggtcagcgattggggtgaagtcgtaacaaggtagccgtaggagaacctgcggctggatcacctcctt

SEQ ID NO: 11 - DNA, 1493 bp, Clostridium glycolicum
agtatcctggctcaggatgaacgctggcggcgtgcctaacacatgcaagtcgagcgattcacttcggtgaagagcggcggacgggtgagtaacgcgtgggtaacctgcctcatacacatggataacataccgaaaggtatgctaatacaggataatataagagattcacatgtatttcttatcaaagctccggcggtatgagatggacccgcgtctgattagctagttggtaaggtaatggcttaccaaggcgacgatcagtagccgacctgagagggtgatcggccacattggaactgagacacggtccaaactcctacgggaggcagcagtggggaatattgcacaatgggcgaaagcctgatgcagcaacgccgcgtgagtgatgaaggccttcgggtcgtaaaactctgtcctcaaggaagataatgacggtacttgaggaggaagccccggctaactacgtgccagcagccgcggtaatacgtagggggctagcgttatccggatttactgggcgtaaagggtgcgtaggtggttttttaagtcaggagtgaaaggctacggctcaaccgtagtaagctcttgaaactgggaaacttgagtgcaggagaggaaagtggaattcctagtgtagcggtgaaatgcgtagatattaggaggaacaccagtagcgaaggcggctttctggactgtaactgacactgaggcacgaaagcgtggggagcgaacaggattagataccctggtagtccacgccgtaaacgatgagtactaggtgtcgggggttacccccctcggtgccgcacgtaacgcattaagtactccgcctggggagtacgctcgcaagagtgaaactcaaaggaattgacggggacccgcacaagtagcggagcatgtggtttaattcgaagcaacgcgaagaaccttacctaagcttgacatccttttgaccgatgcctaatcgcatttttcccttcggggacagaagtgacaggtggtgcatggttgtcgtcagctcgtgtcgtgagatgttgggttaagtcccgcaacgagcgcaacccttgcctttagttgccagcattaagttgggcactctagagggactgccagggataacctggaggaaggtggggatgacgtcaaatcatcatgccccttatgcttagggctacacacgtgctacaatgggtggtacagagggcagccaagtcgtgaggccgagctaatcccttaaatgccattctcagttcggattgtaggctgaaactcgcctacatgaagctggagttactagtaatcgcagatcagaatgctgcggtgaatgcgttcccgggtcttgtacacaccgcccgtcacaccacggaagttgggggcgcccgaagccacttagctaacccttttgggaagcgagtgtcgaaggtgaaatcaataactggggtgaagtcgtaacaaggtagccgtatcggaaggtgcggctggatcacctcctt

SEQ ID NO: 12 - DNA, 20 bp, Artificial
  oligonucleotide useful as primer or propbe
agagtttgatcctggctcag

SEQ ID NO: 13 - DNA, 22 bp, Artificial
  oligonucleotide useful as a probe or primer.
tacggytaccttgttacgactt

SEQ ID NO: 14 - DNA, 20 bp, Artificial
  oligonucleotide useful as a probe or primer.
aaggaggtgatccanccrca

SEQ ID NO: 15 - DNA, 17 bp, Artificial
  oligonucleotide useful as a probe or primer.
accgcttgtgcgggccc

SEQ ID NO: 16 - DNA, 21 bp, Artificial
  oligonucleotide useful as a probe or primer.
catccagtgcaaacctaagag

SEQ ID NO: 17 - DNA, 18 bp, Artificial
  oligonucleotide useful as a probe or primer.
gatccgcttgccttcgca

SEQ ID NO: 18 - DNA, 23 bp, Artificial
  oligonucleotide useful as a probe or primer.
aaaggaagattaatacctcataa

SEQ ID NO: 19 - DNA, 21 bp, Artificial
  oligonucleotide useful as a probe or primer.
taagtttggctccacctcgcg

SEQ ID NO: 20 - DNA, 17 bp, Artificial
  oligonucleotide useful as a probe or primer.
ccaatgtgggggacctt

SEQ ID NO: 21 - DNA, 20 bp, Artificial
  oligonucleotide useful as a probe or primer.
ggaaacaggtgctaataccg

SEQ ID NO: 22 - DNA, 19 bp, Artificial
  oligonucleotide useful as a probe or primer.
ggttagataccgtcaaggg